(19) United States
(12) Patent Application Publication
(10) Pub. No.: US 2017/0318843 A1
(43) Pub. Date: Nov. 9, 2017

(54) ENZYMES FOR MALTING

(71) Applicant: DUPONT NUTRITION BIOSCIENCES APS, Copenhagen (DK)

(72) Inventor: Charlotte Horsmans POULSEN, Brabrand (DK)

(73) Assignee: DuPont Nutrition Biosciences APS, Copenhagen (DK)

(21) Appl. No.: 15/524,603

(22) PCT Filed: Nov. 5, 2015
(86) PCT No.: PCT/EP2015/075842

§ 371 Date: May 4, 2017

(30) Foreign Application Priority Data
Nov. 5, 2014 (EP) 14191889.6

(51) Int. Cl. A23L 7/25; C12C 1/02; C12C 5/00; C12N 9/24; C12N 9/42

(52) U.S. Cl. CPC A23L 7/25; C12N 9/2482; C12N 9/244; C12C 5/004; C12C 1/02; A23V 2002/00

(57) ABSTRACT

The present disclosure provides methods and compositions for preparation of malted cereals, the improved malted cereals and their use, e.g., in the production of food and beverages.

### BACKGROUND

Malting is the process of converting barley and other cereals into malt, typically, for use in brewing, distilling, or in foods. The malting process allows the grain to partially germinate, making the seed's resources available. For example, malting grains develops the enzymes required to modify the grain's starches into sugars, as well as other enzymes, such as proteases, which break down the proteins in the grain into forms that can be used by the fermenting host.

In traditional malting processes, malting process starts with drying the grains to a moisture content below 14%, and then storing for around six weeks to overcome seed dormancy. When ready, the moisture content of cereals is raised by steeping in water several times over a time period, usually several days, to allow the grain to absorb moisture and to start to sprout or germinate. When the grain has a moisture content of around 46%, it is transferred to the germination floor, where the grain is allowed to germinate. The grain at this point is called “green malt”. The green malt is then kilned to the desired specification.

The process of steeping is very water consuming. Typically, to produce 1 liter of malt, the malting process consumes 5 to 10 liters of water. In recent years, there has been a dramatic change in energy and raw material prices caused by increased demand for grains, global water shortage, changing weather factors, etc. Thus, there is a need for improved processes in food production, brewing, and distilling that will bring down costs, increase production efficiency and decrease the amount of water needed.

### SUMMARY OF THE INVENTION

The present disclosure provides compositions and processes for the preparation of malted cereals.

Aspects and embodiments of the compositions and methods are set forth in the following separately numbered paragraphs.

1. A method of producing malted cereal comprising:
a) providing a cereal grain comprising one or more β-glucans,
b) adding to the cereal an effective amount of aβ-glucanase during a malting process; and
c) obtaining a malted cereal.

2. The method of paragraph 1, where the cereal grain further comprises one or more arabinoxylans.

3. In some embodiments of the method of paragraph 2, the method further comprises adding to the cereal an effective amount of a xylanase.

4. In some embodiments of the method of any preceding paragraphs, the method comprises a steeping step including one or more wetting stages, where the β-glucanase and/or the xylanase are added one or more times during the one or more of wetting stages.

5. In some embodiments of the method of paragraph 4, the β-glucanase and/or the xylanase are added at the beginning of the first wetting of the cereal.

6. In some embodiments of the method of paragraph 4, the β-glucanase and/or the xylanase are added during the first wetting of the cereal.

7. In some embodiments of the method of paragraph 4, the β-glucanase and/or the xylanase are added at the beginning of the second wetting of the cereal.

8. In some embodiments of the method of paragraph 4, the β-glucanase and/or the xylanase are added during the second wetting of the cereal.

9. In some embodiments of the method of paragraph 4, the β-glucanase and/or the xylanase are added at the beginning of the third wetting of the cereal.

10. In some embodiments of the method of paragraph 4, the β-glucanase and/or the xylanase are added during the third wetting of the cereal.

11. In some embodiments of the method of paragraph 4, the β-glucanase and/or the xylanase are added at the beginning of all wetting stages of the cereal.

12. In some embodiments of the method of paragraph 4, the β-glucanase and/or the xylanase are added during all wetting stages of the cereal.

13. In some embodiments of the method of paragraph 4, the one or more wetting stages comprise spraying the cereal and where the β-glucanase and/or the xylanase are added one or more times, or constantly, during the spraying.

14. In some embodiments of the method of paragraphs 4-13, the β-glucanase and/or the xylanase are added after a desired temperature has been reached.

15. In some embodiments of the method of any preceding paragraph, the cereal reaches a moisture content of between about 40 to about 50% W/W.

16. In some embodiments of the method of paragraph 15, the moisture content is reached in less than 12 hours.

17. In some embodiments of the method of paragraph 15 or 16, the moisture content is reached with at least 10% less water than the water used for a malted cereal prepared without the β-glucanase and/or the xylanase.

18. In some embodiments of the method of any preceding paragraph, the cereal has 1-10% W/W of β-glucans.

19. In some embodiments of the method of any preceding paragraph, the cereal has 1-10% W/W of arabinoxylans.

20. In some embodiments of the method of any preceding paragraph, the amount of high molecular weight β-glucans in the cereal is decreased at least 50%.

21. In some embodiments of the method of any preceding paragraph, the amount of high molecular weight β-glucans in the cereal is decreased at least 80%.

22. In some embodiments of the method of paragraphs 3-20, where of the amount of high molecular weight arabinoxylans in the cereal is decreased at least 50%.

23. In some embodiments of the method of any preceding paragraph, the cereal is selected from the group consisting of barley, wheat, rye, corn, oats, rice, millet, triticale, cassava, sorghum and a combination thereof.

24. In some embodiments of the method of any preceding paragraph, the β-glucanase is dosed at 0.01-100 mg enzyme protein per kilogram of cereal.

25. In some embodiments of the method of any preceding paragraph the β-glucanase is dosed at 1-15 mg enzyme protein per kilogram of cereal.

26. In some embodiments of the method of any preceding paragraph, the xylanase is dosed at 0.01-100 mg enzyme protein per kilogram of cereal.

27. In some embodiments of the method of any preceding paragraph, the xylanase is dosed at 1-15 mg enzyme protein per kilogram of cereal.

28. In some embodiments of the method of any preceding paragraph, the β-glucanase is an endo-1,3(4)-β-glucanase.

29. In some embodiments of the method of paragraph 28, the endo-1,3(4)-β-glucanase enzyme comprises an amino acid sequence having at least 80% identity with any one selected from SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof

30. In some embodiments of the method of paragraphs 3-29, the xylanase is an endo-1,4-β-xylanase.

31. In some embodiments of the method of paragraph 30 the endo-1,4-β-xylanase enzyme comprises an amino acid sequence having at least 80% identity with any one selected from SEQ ID NO:1, SEQ ID NO:2, SEQ ID NO:3, SEQ ID NO:4, SEQ ID NO:5, SEQ ID NO:6, SEQ ID NO:17, and SEQ ID NO:18, or any functional fragment thereof.

32. In some embodiments of the method of paragraphs 3-31, the β-glucanase and the xylanase have an amino acid sequence having at least 80% sequence identity with the respective SEQ ID, or any functional fragment thereof, being selected from the list consisting of SEQ ID NO:1 and SEQ ID NO:7; SEQ ID NO:2 and SEQ ID NO:7; SEQ ID NO:3 and SEQ ID NO:7; SEQ ID NO:4 and SEQ ID NO:7; SEQ ID NO:5 and SEQ ID NO:7; SEQ ID NO:6 and SEQ ID NO:7; SEQ ID NO:17 and SEQ ID NO:7; SEQ ID NO:18 and SEQ ID NO:7; SEQ ID NO:1 and SEQ ID NO:8; SEQ ID NO:2 and SEQ ID NO:8; SEQ ID NO:3 and SEQ ID NO:8; SEQ ID NO:4 and SEQ ID NO:8; SEQ ID NO:5 and SEQ ID NO:8; SEQ ID NO:6 and SEQ ID NO:8; SEQ ID NO:17 and SEQ ID NO:8; SEQ

ID NO:18 and SEQ ID NO:8; SEQ ID NO:1 and SEQ ID NO:9; SEQ ID NO:2 and SEQ ID NO:9; SEQ ID NO:3 and SEQ ID NO:9; SEQ ID NO:4 and SEQ ID NO:9; SEQ ID NO:5 and SEQ ID NO:9; SEQ ID NO:6 and SEQ ID NO:9; SEQ ID NO:17 and SEQ ID NO:9; SEQ ID NO:18 and SEQ ID NO:9; SEQ ID NO:1 and SEQ ID NO:10; SEQ ID NO:2 and SEQ ID NO:10; SEQ ID NO:3 and SEQ ID NO:10; SEQ ID NO:4 and SEQ ID NO:10;

SEQ ID NO:5 and SEQ ID NO:10; SEQ ID NO:6 and SEQ ID NO:10; SEQ ID NO:17 and SEQ ID NO:10; SEQ ID NO:18 and SEQ ID NO:10; SEQ ID NO:1 and SEQ ID NO:11; SEQ ID NO:2 and SEQ ID NO:11; SEQ ID NO:3 and SEQ ID NO:11; SEQ ID NO:4 and SEQ ID NO:11; SEQ ID NO:5 and SEQ ID NO:11; SEQ ID NO:6 and SEQ ID NO:11; SEQ ID NO:17 and SEQ ID NO:11; SEQ ID NO:18 and SEQ ID NO:11; SEQ ID NO:1 and SEQ ID NO:12; SEQ ID NO:2 and SEQ ID NO:12; SEQ ID NO:3 and SEQ ID NO:12; SEQ ID NO:4 and SEQ ID NO:12; SEQ ID NO:5 and SEQ ID NO:12; SEQ ID NO:6 and SEQ ID NO:12; SEQ ID NO:17 and SEQ ID NO:12; SEQ ID NO:18 and SEQ ID NO:12; SEQ ID NO:1 and SEQ ID NO:13; SEQ ID NO:2 and SEQ ID NO:13; SEQ ID NO:3 and SEQ ID NO:13; SEQ ID NO:4 and SEQ ID NO:13; SEQ ID NO:5 and SEQ ID NO:13; SEQ ID NO:6 and SEQ ID NO:13; SEQ ID NO:17 and SEQ ID NO:13; SEQ ID NO:18 and SEQ ID NO:13; SEQ ID NO:1 and SEQ ID NO:14; SEQ ID NO:2 and SEQ ID NO:14; SEQ ID NO:3 and SEQ ID NO:14; SEQ ID NO:4 and SEQ ID NO:14; SEQ ID NO:5 and SEQ ID NO:14; SEQ ID NO:6 and SEQ ID NO:14; SEQ ID NO:17 and SEQ ID NO:14; SEQ ID NO:18 and SEQ ID NO:14; SEQ ID NO:1 and SEQ ID NO:15; SEQ ID NO:2 and SEQ ID NO:15; SEQ ID NO:3 and SEQ ID NO:15; SEQ ID NO:4 and SEQ ID NO:15; SEQ ID NO:5 and SEQ ID NO:15; SEQ ID NO:6 and SEQ ID NO:15; SEQ ID NO:17 and SEQ ID NO:15; SEQ ID NO:18 and SEQ ID NO:15; SEQ ID NO:1 and SEQ ID NO:16; SEQ ID NO:2 and SEQ ID NO:16; SEQ ID NO:3 and SEQ ID NO:16; SEQ ID NO:4 and SEQ ID NO:16; SEQ ID NO:5 and SEQ ID NO:16; SEQ ID NO:6 and SEQ ID NO:16; SEQ ID NO:17 and SEQ ID NO:16; and SEQ ID NO:18 and SEQ ID NO:16.

33. In some embodiments of the method of paragraphs 3-31, the β-glucanase and the xylanase have an amino acid sequence having at least 80% sequence identity with the respective SEQ ID, or any functional fragment thereof, being selected from the list consisting of SEQ ID NO:1 and SEQ ID NO:7; SEQ ID NO:2 and SEQ ID NO:8; and SEQ ID NO:2 and SEQ ID NO:8.

34. In some embodiments of the method of paragraphs 3-33, the xylanase and the beta-glucanase are combined at a ratio of 1:1 per weight.

35. In some embodiments of the method of paragraphs 3-34, the beta-glucanase has a beta-glucanase activity of at least 9000 U/g, and the xylanase has a xylanase activity of at least 13000 U/g.

36. In some embodiments of the method of paragraph 3-31 the β-glucanase and the xylanase comprises commercially available enzymes having xylanase activity and/or beta-glucanase activity selected from the group consisting of UltraFlo L (available from Novozymes-beta glucanase with cellulase, xylanase side activities), UltraFlo XL (available from Novozymes-beta glucanase with xylanase and alpha amylase side activities), UltraFlo Max (available from Novozymes-beta glucanase and xylanase), Finizyme 250 L(available from Novozymes-beta glucanase with cellulase, xylanase side activities) and Filtrase series(available from DSM-betaglucanase and xylanases).

37. In some embodiments of the method according to any preceding paragraph, the further comprises adding one or more enzymes selected from the group of amylase, a protease, naturally occurring enzymes in the cereal and combinations thereof.

38. In some embodiments of the method according to any preceding paragraph, a total steeping time is at least 10% less than the time of a malted cereal prepared without the β-glucanase and/or the xylanase.

39. In some embodiments of the method according to any preceding paragraph, a total steeping time does not exceed 25 hours.

40. In some embodiments of the method according to any preceding paragraph, a total steeping time does not exceed 12 hours.

41. In some embodiments of the method according to any preceding paragraph, a total amount of water used during the steeping process is at least 10% less than the water used for a malted cereal prepared without the β-glucanase and/or the xylanase.

42. In some embodiments of the method according to any preceding paragraph, the amount of high molecular weight β-glucans in the malted cereal is at least 10% less than the amount of high molecular weight β-glucans in a malted cereal prepared without the β-glucanase.

43. In some embodiments of the method of paragraphs 3-42, the amount of high molecular weight arabinoxylans is at least 10% less than the amount of arabinoxylans in a malted cereal prepared without the xylanase.

44. In some embodiments of the method of paragraphs 3-42, the amount of high molecular weight β-glucans and arabinoxylans is at least 50% less than the amount of β-glucans and/or arabinoxylans in a malted cereal prepared without the β-glucanase and/or the xylanase.

45. In some embodiments of the method of paragraphs 2-43, the amount of high molecular weight β-glucans in the malted cereal is 50 mg/l or less, and the amount of arabinoxylans in the malted cereal is at 2000 mg/l or less.

46. In some embodiments of the method of any preceding paragraph, an extract prepared from the malted cereal has lower viscosity that an extract prepared with a malted cereal prepared without the β-glucanase and/or the xylanase.

47. In some embodiments of the method according to any preceding paragraph, the malted cereal has increased enzymatic activity.

48. In some embodiments of the method of paragraph 47, the enzymatic activity is increased by a factor of at least about 2 as compared to a malted cereal prepared without the β-glucanase and/or the xylanase.

49. In some embodiments of the method of paragraphs 47 or 48, the enzymatic activity is selected from the group consisting ofβ-glucanase activity, xylanase activity, amylase activity, protease activity, naturally occurring enzyme activity in the cereal and combinations thereof.

50. The method of paragraph 47 or 48, wherein the enzymatic activity is β-glucanase and/or xylanase activity.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 shows a schematic representation of the malting process. Enzymes described herein can be added one or more times, or constantly, during one or more step of the process.

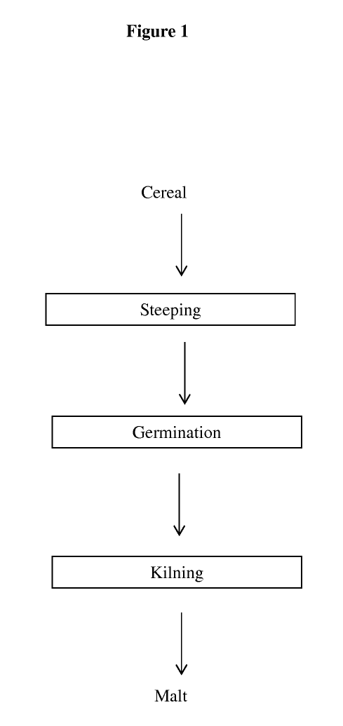

### DETAILED DESCRIPTION OF THE INVENTION

The present disclosure provides compositions and processes for the preparation of malted cereals, e.g., for the use in production of food and beverages. In some embodiments, the present invention provides methods and compositions for improved preparation of malted cereals, e.g., reduced water consumption and/or germination time. In some embodiments, the present invention provides methods and compositions for improved preparation of malted cereals having higher enzymatic activity. In some embodiments, the present invention provides methods and compositions for improved preparation of malted cereals which improve the processes for food and beverage production, and malted cereals that may improve the properties of said obtained food and beverages.

In one aspect, the present invention is directed to composition and methods for the preparation of malted cereals comprising adding one or more enzymes to the malting process. In some embodiments, the present invention provides compositions and methods for the preparation of malted cereals comprising adding a cell wall degrading enzyme alone or in combination with other enzymes at some point during the malting process. In some embodiments, the cell wall degrading enzyme is a xylanase or a beta glucanase. In one embodiment, the present invention provides compositions and methods for the preparation of malted cereals comprising adding a beta glucanase alone or in combination with other enzymes at some point during the malting process. In one embodiment, the present invention provides compositions and methods for the preparation of malted cereals comprising adding a xylanase alone or in combination with other enzymes at some point during the malting process. In some embodiments, the other enzymes are other cell wall degrading enzymes. In some embodiments the invention provides compositions and methods for the preparation of malted cereals comprising adding a beta glucanase and a xylanase alone or in combination with other enzymes during the malting process. Examples of cereals that can be used in the methods described herein include but are not limited to barley, wheat, rye, corn, oats, rice, millet triticale, cassava, sorghum and the like.

Cereals, such as barley, wheat, rye, corn, oats, rice, millet triticale, cassava, and sorghum, used in the production of food and beverages (e.g. beer) contain varying levels of β-glucans and arabinoxylans. (1,3;1,4)-β-D-Glucans consist of unbranched and unsubstituted chains of (1,3)- and (1,4)-β-glucosyl residues. The (1,3;1,4)-β-D-glucans are most abundant in cell walls of the cereals, specifically in the starchy endosperm of grain, where they can contribute up to 70% by weight of the cell walls in barley, rye, and oats. Arabinoxylan is a hemicellulose found in both the primary and secondary cell walls of cereal grains, consisting of copolymers of two pentose sugars-arabinose and xylose. The arabinose moiety may be further substituted.

Studies have shown that the content of β-glucans and arabinoxylans in the cell wall might be related with grain hardness as well as water uptake of the cereals (e.g. barley). Kernel hardness is one of the major factors affecting the processing and product quality of the grain. A high β-glucans and arabinoxylans content in the cereal may lead to insufficient degradation of cell walls, which in turns hinders the diffusion of germination enzymes and the mobilization of kernel reserves, and hence reduces the malt extract. Further studies have suggested that that residual β-glucans may lead to highly viscous wort, giving rise to filtration problems, and it may participate in causing chill haze.

Without intending to be limited to any theory, in some embodiments, the present invention provides compositions and methods to break down betaglucans and other cell wall components such as arabinoxylans in the grains during the malting process. By breaking down the betaglucans and other cell wall components (e.g., arabinoxylans) in the grain during the malting process the present invention allows for usage of less water during the malting process by decreasing the water binding capacity of beta-glucans and arabinoxylans in the cell wall of the grain while allowing a better and faster water uptake by the grain. Further by breaking down the beta-glucans and other cell wall components (e.g., arabinoxylans) in the grain, the present invention allows for better diffusion of one or more enzymes in the cereal and for better substrate accessibility for said enzymes. Thus, in some embodiments, the present invention provides composition and methods to decrease the amount of water needed during malting (e.g. for steeping). In some embodiments, the present invention provides composition and methods to improve the water uptake by the grain. Further, in some embodiments, the present invention provides composition and methods to decrease the time needed for steeping and the time needed to cause the grains to germinate. In some embodiments, the present invention provides methods and compositions for improved preparation of malted cereals having higher enzymatic activity.

In some embodiments, the preparation process of malted cereals comprises the following steps: a steeping step including one or more wetting stages, a germination step, a drying step (e g kilning) including one or more temperature, and one or more enzymes as described herein which are added one or more times during the process. In some embodiments, the same or different one or more enzymes are added in one or more of the wetting stages of the steeping process.

The invention further provides composition and methods for improved preparation of malted cereals having higher enzymatic activity (e.g. betaglucanase and xylanase activity), which can then improve downstream processes for food and beverage production (e.g. easier and more effective processing of the malt in wort and beer production). Thus, in some embodiments, the invention provides enzymes suitable for the production of food and beverage products, such as in the production of an alcoholic or non-alcoholic beverage, such malt-based beverage like beer or whiskey, malted shakes, malt vinegar, flavored drinks such as Malta, Horlicks, Ovaltine and Milo, and in the production of confections such as Maltesers and Whoppers, and some baked goods, such as malt loaf, bagels and rich tea biscuits. The improved properties of the malted cereals comprise e.g. improved temperature optimums, improved ratio in activity on soluble (WE-AX) to insoluble (WU-AX) arabinoxylan substrates, reduced total pressure built up during mash separation and/or filtration steps of a brewing process, as well as increased filterability of enzyme treated material.

As used herein, the terms “beverage” and “beverage(s) product” includes such foam forming fermented beverages as beer brewed with 100% malt, beer brewed under different types of regulations, ale, dry beer, near beer, light beer, low alcohol beer, low calorie beer, porter, bock beer, stout, malt liquor, non-alcoholic beer, non-alcoholic malt liquor and the like. The term “beverages” or “beverages product” also includes non-foaming beer and alternative malt beverages such as fruit flavored malt beverages, e. g. , citrus flavored, such as lemon-, orange-, lime-, or berry-flavored malt beverages, liquor flavored malt beverages, e. g. , vodka-, rum-, or tequila-flavored malt liquor, or coffee flavored malt beverages, such as caffeine-flavored malt liquor, and the like.

Beer is traditionally referred to as an alcoholic beverage derived from malt, such as malt derived from barley grain, and optionally adjunct, such as starch containing plant material (e.g. cereal grains) and optionally flavored, e.g. with hops. In the context of the present invention, the term “beer” includes any fermented wort, produced by fermentation/brewing of a starch-containing plant material, thus in particular beer produced from malt and beer produced from any combination of malt and adjunct. Beer can be made from a variety of starch-containing plant material by essentially the same process, where the starch consists mainly of glucose homopolymers in which the glucose residues are linked by alpha-1, 4- or alpha-1,6-bonds, with the former predominating.

The term “germination” or “germinate” or any other equivalent as used herein means the beginning or resumption of growth by a seed. In accordance with the processes described herein, germination begins to occur during and/or after the cereal has been steeped. Germination of cereals is generally understood to mean hydration of the seed, swelling of the cereal and inducing growth of the embryo. Environmental factors affecting germination include moisture, temperature and oxygen level. A rapid increase in cells of the root stem leads to root development, while corresponding growth sends forth a shoot.

As used herein, the term “steeping” refers to wetting of the cereal. Wetting may include one or more stages over a time and temperature effective for providing a moisture content desired for a malting process, e.g., between about 20% and about 60% by weight, or between 40% to about 50%. Wetting may also including constant spraying of the cereal, or spraying at one or more stages during the malting process (e.g., during the germination step).

Unless defined otherwise, all technical and scientific terms used herein have the same meaning as is commonly understood by one of skill in the art to which this invention belongs. All patents and publications referred to herein are incorporated by reference.

Enzymes

In one aspect, the present invention is directed to composition and methods for the preparation of malted cereals comprising adding one or more enzymes to the malting process. In one embodiment, the present invention provides compositions and methods for the preparation of malted cereals comprising adding a beta glucanase and/or a xylanase alone or in combination with other enzymes during the malting process.

In one embodiment, the present invention provides compositions and methods for the preparation of malted cereals comprising adding a beta glucanase alone or in combination with other enzymes during the malting process.

In some embodiments, the enzyme for the methods and compositions described herein may have cellulolytic activity. The systematic name of cellulose is (1,3;1,4)-beta-D-glucan. 4-glucanohydrolase and cellulolytic enzymes or cellulases are classified in EC 3.2.1.4. Cellulases endohydrolyse (1 4)- beta- D-glucosidic linkages (in, e.g., cellulose, and lichenin), beta-D-glucans, and will also hydrolyse 1,4-linkages in β-D-glucans (also containing 1,3-linkages). Cellulase also have other names such as endo-1,4-beta-D-glucanase, beta-1,4-glucanase, beta-1,4-endoglucan hydrolase, cellulase A, cellulosin AP, endoglucanase D, alkali cellulose, cellulase A 3, celludextrinase, 9.5 cellulase, avicelase, pancellase SS and 1,4-(1,3;1,4)-beta-D-glucan 4-glucanohydrolase.

In one aspect of the invention, the cellulase activity of the enzyme composition according to the invention can be measured by the cellulase activity assays as described herein or by any feasible method known in the art.

In further aspects, the present invention relates to enzymes having endo-1,3(4)-beta-glucanase activity, which can be determined by the assays described herein or by any feasible method known in the art.

β-glucanase” or “beta-glucanase” as used herein refers to an endo-1,3(4)-beta-glucanase of EC 3.2.1.6. β-glucanases catalyze the endohydrolysis of (1→3)- or (1→4)-linkages in beta-D-glucans. Suitable beta-glucanases to be used alone or in combination with one or more cell wall degrading enzyme (e.g., an enzyme exhibiting endo-1,4-beta-xylanase activity) according to the invention includes any one betaglucanase disclosed in WO2004087889, WO2005059084, WO9414953, WO2007056321, WO9531533, WO08023060, WO2005100582, WO9828410, WO9742301, WO2006066582, WO05118769, WO2005003319, and WO10059424.

Typically, standard assays are carried out at pH 5.0, however, the assays can be performed at different pH values for the additional characterization and specification of enzymes.

One unit of endo-1,3(4)-beta-glucanase activity is defined as the amount of enzyme which produces 1 μmole glucose equivalents per minute under the conditions of the assay (e.g., pH 5.0 (or as specified) and 50° C.).

In some embodiments, the enzyme for the methods and compositions described herein comprises β-glucanase activity of at least about 10000 U/g, such as at least about 12000 U/g, such as at least about 14000 U/g, such as at least about 15000 U/g, such as at least about 18000 U/g as measured by the assays described herein or by any feasible method known in the art. In some embodiments, a different β-glucanase activity is used depending of the β-glucan content in the cereal and/or the malting conditions and/or the malted cereal requirements.

In further aspects, the enzyme for the methods and compositions described herein has laminarinase activity or comprises any one or more further enzyme having laminarinase activity. The laminarinase activity can be determined as described in the laminarase assays herein or by any feasible method known in the art.

Laminarinase may be an endo-1,3(4)-beta-glucanase classified in E.C. 3.2.1.6 or glucan endo-1,3-beta-D-glucosidase classified in E.C. 3.2.1.39. Endo-1,3(4)-beta-glucanase with the alternative names, laminarinase, endo-1,3-beta-glucanase. Endo-1,4-beta-glucanase is classified in E.C. 3.2.1.6. The substrates include laminarin, lichenin and cereal D-glucans. The enzyme catalyzes endohydrolysis of (1→3)- or (1→4)-linkages in beta-D-glucans when the glucose residue whose reducing group is involved in the linkage to be hydrolyzed is itself substituted at C-3. Glucan endo-1,3-beta-D-glucosidase with the alternative names (1→3)-beta-glucan endohydrolase, Endo-1,3-beta-glucanase and laminarinase is classified in E.C. 3.2.1.39. Glucan endo-1,3-beta-D-glucosidase hydrolyses (1→3)-beta-D-glucosidic linkages in (1→3)-beta-D-glucans in substrates as e.g. laminarin, paramylon and pachyman.

In some aspects, the enzyme for the methods and compositions described herein has xyloglucan-specific exo-beta-1,4-glucanase activity or comprises a further enzyme having xyloglucan-specific exo-beta-1,4-glucanase activity, “xyloglucan-specific exo-beta-1,4-glucanase” refers to enzymes of E.C3.2.1.155. Xyloglucan-specific exo-beta-1,4-glucanase catalyze the exohydrolysis of (1→4)-beta-D-glucosidic linkages in xyloglucan.

In one embodiment, the present invention provides compositions and methods for the preparation of malted cereals comprising adding a beta glucanase alone or in combination with other enzymes during the malting process. In some embodiments, the other enzymes are cell wall degrading enzymes. In some embodiments, the cell wall degrading enzyme is a xylanase. Thus, in some embodiments the invention provides compositions and methods for the preparation of malted cereals comprising adding a beta glucanase and a xylanase alone or in combination with other enzymes during the malting process.

In one embodiment, the present invention provides compositions and methods for the preparation of malted cereals comprising adding a xylanase alone or in combination with other enzymes during the malting process. In some embodiments, the other enzymes are cell wall degrading enzymes.

Xylanases are classified in EC 3.2.1.8, EC 3.2.1.32, EC 3.2.1.136 and EC 3.2.1.156.; their activity may be measured e.g. as described herein or by any suitable method known in the art. Suitable xylanases to be used in combination with an enzyme exhibiting endo-1,3(4)-beta-glucanase activity according to the invention includes any xylanase classified in EC 3.2.1.8, EC 3.2.1.32, EC 3.2.1.136 and EC 3.2.1.156, such as any of the ones disclosed in WO 2010072226, WO 2010072225, WO2010072224, WO 2005059084, WO2007056321, WO2008023060A, WO9421785, WO2006114095, WO2006066582, US 2008233175, and W010059424.

Endo-1,4-beta xylanase is classified as EC 3.2.1.8. The enzyme causes endohydrolysis of 1,4-beta-D-xylosidic linkages in xylans.

The terms “family 11 xylanase”, “Glycoside hydrolase (GH) family 11” or simply “GH 11 xylanase” as used herein refers to an endo-1,4-beta xylanase classified as EC 3.2.1.8, which causes endohydrolysis of 1,4-beta-D-xylosidic linkages in xylans and which is classified as a family 11 xylanase according to B. Henrissat , A classification of glycosyl hydrolases based on amino acid sequence similarities. Biochem. J. 280 (1991), pp. 309-316.

The terms “Family 10 xylanase”, “Glycoside hydrolase (GH) family 10”, or simply “GH 10 xylanase” comprises enzymes with a number of known activities, such as xylanase (EC:3.2.1.8); endo-1,3-beta-xylanase (EC:3.2.1.32); cellobiohydrolase (EC:3.2.1.91). These enzymes were formerly known as cellulase family F.

In some embodiments the enzyme exhibiting endo-1,4-beta-xylanase activity is a family 11 xylanase. In some embodiments the enzyme exhibiting endo-1,4-beta-xylanase activity is a family 10 xylanase.

In some embodiments, the enzyme exhibiting xylanase activity is a family 5 xylanase. The term “Family 5 xylanase” (formerly family A) includes glycoside hydrolases with varying activities, including: endoglycosylceramidase (EC 3.2.1.123), cellulase (EC 3.2.1.4), licheninase (EC3.2.1.73), beta-mannosidase (EC 3.2.1.25), glucan 1,3-bglucosidase (EC 3.2.1.58), glucan endo-1,6-beta-glucosidase (EC 3.2.1.75), mannan endo-1,4-beta-mannosidase (EC 3.2.1.58), cellulose 1,4-beta-cellobiosidase (EC3.2.1.91), endo-1,6-beta-galactanase (EC 3.2.1.-), 1,3-mannanase (EC 3.2.1.-) and endo-1,4-beta-xylanase (EC3.2.1.8).

In one aspect, the enzyme composition according to the invention has endo-1,4-beta xylanase activity as measured by the assays described herein or any suitable assay known in the art.

An assay for measuring xylanase activity may be carried out at pH 3.5 or pH 5 and 50° C. using xylan as substrate, or it can be performed at different pH and temperature values for the additional characterization and specification of enzymes. Enzyme activity can be calculated from the increase in absorbance caused by xylose at 540 nm per unit time.

In some embodiments the enzyme composition according to the invention comprises a xylanase activity of at least about 5000 U/g, such as at least about 6000 U/g, such as at least about 7000 U/g, such as at least about 8000 U/g, such as at least about 8500 U/g, as measured by in the assays described herein or any suitable assay known in the art. In some embodiments, a different 13-xylanase activity is used, e.g., depending of the arabinoxylan content in the cereal and/or the malting conditions and/or the malted cereal requirements.

In some aspects, the enzyme composition according to the invention has arabinanase activity or comprises a further enzyme having arabinanase activity. Arabinanase is classified as EC3.2.1.99. The systematic name is 5-alphα-L-arabinan 5-alphα-L-arabinanohydrolase but it has several other names such as arabinan endo-1,5-alphα-L-arabinosidase, and endo-1,5-alphα-L-arabinanase, endo-alpha-1,5-arabanase, endo-arabanase, 1,5-alphα-L-arabinan and 1,5-alphα-L-arabinanohydrolase. Arabinase endohydrolyses (1→5)-alpha-arabinofuranosidic linkages in (1→5)-arabinans. Arabinanase also acts on arabinan.

In one aspect of the invention, the arabinase activity of the enzyme composition according to the invention is measured by arabinase assay as herein or any suitable method known in the art. The assay can be carried out at pH 3.5 and 50° C. using sugar beet arabinan as substrate, and it can be performed at different pH and temperature values for the additional characterization and specification of enzymes. Enzyme activity can be calculated from the increase in absorbance at 540 nm per unit time.

One unit of arabinase activity is defined as the amount of enzyme (normalized for total assay volume) that gives an increase in absorbance under the conditions of the assay used (e.g., pH 3.5 and 50° C.).

In some aspects, the enzyme composition according to the invention has beta-D-glucoside glucohydrolase activity or comprises a further enzyme having beta-D-glucoside glucohydrolase activity. Beta-D-glucoside glucohydrolase refers to enzymes of E.C 3.2.1.21.

In some aspects, the enzyme composition according to the invention has β-Xylosidase activity or comprises a further enzyme having 13-Xylosidase activity. “β-Xylosidase” or “Xylan 1,4-beta-xylosidase” refers to enzymes of E.0 3.2.1.37. β-Xylosidase catalyze the hydrolysis of (1→4)-beta-D-xylans, to remove successive D-xylose residues from the non-reducing termini.

In some aspects of the invention, the enzyme composition according to the invention has cellobiohydrolase activity or comprises a further enzyme having cellobiohydrolase activity. “Cellobiohydrolase” or “Cellulose 1,4-beta-cellobiosidase” refers to enzymes of EC 3.2.1.91. Cellulose 1,4-beta-cellobiosidase catalyzes hydrolysis of 1,4-beta-D-glucosidic linkages in cellulose and cellotetraose, releasing cellobiose from the non-reducing ends of the chains.

The cellobiohydrolase activity of the enzyme composition according to the invention is measured by the cellobiohydrolase assays as described herein or any suitable assay known in the art. The standard assay is carried out at pH 5.0, and it can be performed at different pH values for the additional characterization and specification of enzymes.

One unit of cellobiohydrolase activity is defined as the amount of enzyme which produces 1 μmole p-nitrophenol from p-nitrophenyl b-D-cellobiopyranoside per minute under the conditions of the assay (e.g., pH 5.0 (or as specified) and 50° C.).

In some aspects, the enzyme composition according to the invention has alpha-N-arabinofuranosidase activity or comprises a further enzyme having arabinofuranosidase activity. “alpha-N-arabinofuranosidase” or “Alpha-N-arabinofuranosidase” refers to enzymes of EC 3.2.1.55. alpha-N-arabinofuranosidase catalyzes the hydrolysis of terminal non-reducing alphα-L-arabinofuranoside residues in alphα-L-arabinosides.

In one aspect of the invention, the arabinofuranosidase activity of the enzyme composition according to the invention is measured by the arabinofuranosidase assay as described herein or by any suitable assay known in the art. The standard assay can be carried out at pH 5.0 and 50° C. and it can be performed at different values of pH and temperatures for the additional characterization and specification of enzymes.

One unit of alpha-N-arabinofuranosidase activity is defined as the amount of enzyme which produces 1 μmole p-nitrophenol from p-nitrophenyl alphα-L-arabinofuranoside per minute under the conditions of the assay (e.g., pH 5.0 and 50° C. (or as specified)).

In some aspects, the enzyme composition according to the invention has glucan 1,4-betaglucosidase activity or comprises a further enzyme having glucan 1,4-beta-glucosidase activity. “Glucan 1,4-beta-glucosidase” or “glucan 1,4-beta-glucosidase” refers to enzymes of E.C3.2.1.74. Glucan 1,4-beta-glucosidase catalyze the hydrolysis of (1→4)-linkages in (1→4)-beta-D-glucans, to remove successive glucose units.

In some embodiments, one or more enzyme exhibiting endo-1,4-beta-xylanase activity and/or one or more enzyme exhibiting beta-glucanase activity, alone or in combination, provide for improved malting processes.

In some embodiments, one or more enzyme exhibiting endo-1,4-beta-xylanase activity and/or one or more enzyme exhibiting beta-glucanase activity, alone or in combination, provide for a decrease in water consumption during the steeping process.

In some embodiments, one or more enzyme exhibiting endo-1,4-beta-xylanase activity and/or one or more enzyme exhibiting beta-glucanase activity, alone or in combination, provide for improved and/or faster intake of water by the grain during the steeping process.

In some embodiments, one or more enzyme exhibiting endo-1,4-beta-xylanase activity and/or one or more enzyme exhibiting beta-glucanase activity, alone or in combination, provide a decrease in the time needed for steeping.

In some embodiments, one or more enzyme exhibiting endo-1,4-beta-xylanase activity and/or one or more enzyme exhibiting beta-glucanase activity, alone or in combination, provide for a decrease in the time needed to cause the grains to germinate.

In some embodiments, one or more enzyme exhibiting endo-1,4-beta-xylanase activity and/or one or more enzyme exhibiting beta-glucanase activity, alone or in combination, provide for a malted cereal with increase enzymatic activity (e.g., xylanase activity and/or beta-glucanase activity).

In some embodiments, one or more enzyme exhibiting endo-1,4-beta-xylanase activity and/or one or more enzyme exhibiting beta-glucanase activity, alone or in combination, provide for better diffusion of one or more cereal enzymes and for better substrate accessibility for said enzymes.

In some embodiments, one or more enzyme exhibiting endo-1,4-beta-xylanase activity and/or one or more enzyme exhibiting beta-glucanase activity, alone or in combination, provide for a malted cereal with a decrease amount of high molecular weight β-glucans and arabinoxylans.

In some embodiments, one or more enzyme exhibiting endo-1,4-beta-xylanase activity and/or one or more enzyme exhibiting beta-glucanase activity, alone or in combination, provide for a malted cereal with lower viscosity.

In some embodiments, one or more enzyme exhibiting endo-1,4-beta-xylanase activity and/or one or more enzyme exhibiting beta-glucanase activity, alone or in combination, provide for a malted cereal with a lower potential for off flavor formation, such as off flavor formation related to arabinoxylan breakdown.

In some embodiments, one or more enzyme exhibiting endo-1,4-beta-xylanase activity and/or one or more enzyme exhibiting beta-glucanase, alone or in combination, provide for a malted cereal with better filterability, such as mash separation and beer filtration.

One aspect of the invention relates to an enzyme exhibiting endo-1,4-beta-xylanase activity, which enzyme comprises an amino acid sequence having at least 80% identity with any one selected from SEQ ID NO:1, SEQ ID NO:2, SEQ ID NO:3, SEQ ID NO:4, SEQ ID NO:5, SEQ ID NO:6, SEQ ID NO:17, and SEQ ID NO:18 , or any functional fragment thereof.

As used herein “functional fragment” refers to a truncated version of an enzyme with essentially the same or at least a significant degree of enzyme activity as the non-truncated reference enzyme.

Another aspect relates to an enzyme exhibiting endo-1,3(4)-beta-glucanase activity, which enzyme comprises an amino acid sequence having at least 80% identity with any one selected from SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof.

In some embodiments, the enzyme has a temperature optimum in the range of 5-80° C., such as in the range of 5-40° C. or 15-80° C., such as in the range 20-80° C., such as in the range 5-15° C., 15-20° C., 45-65° C., 50-65° C., 55-65° C. or 60-80° C.

In some embodiments, the enzyme has at least 81, 82, 83, 84, 85, 86, 87, 88, 89, 90, 91, 92, 93, 94, 95, 96, 97, 98 or 99% identity with any one amino acid sequence selected from SEQ ID NO: 1-18, or any functional fragment thereof.

In some embodiments, the enzyme has a total number of amino acids of less than 350, such as less than 340, such as less than 330, such as less than 320, such as less than 310, such as less than 300 amino acids, such as in the range of 200 to 350, such as in the range of 220 to 345 amino acids.

In some embodiments, the amino acid sequence of the enzyme has at least one, two, three, four, five, six, seven, eight, nine or ten amino acid substitutions as compared to any one amino acid sequence selected from SEQ ID NO: 1-18, or any functional fragment thereof.

In some embodiments, the amino acid sequence of the enzyme has a maximum of one, two, three, four, five, six, seven, eight, nine or ten amino acid substitutions compared to any one amino acid sequence selected from SEQ ID NO: 1-18, or any functional fragment thereof.

In some embodiments, the enzyme comprises the amino acid sequence identified by any one of SEQ ID NO: 1-18, or any functional fragment thereof

In some embodiments, the enzyme consists of the amino acid sequence identified by any one of SEQ ID NO: 1-18, or any functional fragment thereof.

A further important aspect of the invention relates to compositions and methods comprising an enzyme exhibiting endo-1,4-p -xylanase activity in combination with any one or more beta-glucanase as described herein.

A further important aspect of the invention compositions and methods comprising an enzyme exhibiting endo-1,3(4)-beta-glucanase activity in combination with any one or more xylanase as described herein. In some embodiments this one or more xylanase is an enzyme according to SEQ ID NO: 17 and/or SEQ ID NO: 18, or any functional fragment thereof.

In some embodiments the combination of an enzyme exhibiting endo-1,4-beta-xylanase activity with an enzyme exhibiting endo-1,3(4)-beta-glucanase activity is according to the following table:

| | Xylanase | | | | | | | |
|---|---|---|---|---|---|---|---|---|
| | | | | | | | | SEQ |
| Glucanase | SEQ ID NO: 1 | SEQ ID NO: 2 | SEQ ID NO: 3 | SEQ ID NO: 4 | SEQ ID NO: 5 | SEQ ID NO: 6 | SEQ ID NO: 17 | ID NO: 18 |
| SEQ ID NO: 7 | X | X | X | X | X | X | X | X |
| SEQ ID NO: 8 | X | X | X | X | X | X | X | X |
| SEQ ID NO: 9 | X | X | X | X | X | X | X | X |
| SEQ ID NO: 10 | X | X | X | X | X | X | X | X |
| SEQ ID NO: 11 | X | X | X | X | X | X | X | X |
| SEQ ID NO: 12 | X | X | X | X | X | X | X | X |
| SEQ ID NO: 13 | X | X | X | X | X | X | X | X |
| SEQ ID NO: 14 | X | X | X | X | X | X | X | X |
| SEQ ID NO: 15 | X | X | X | X | X | X | X | X |
| SEQ ID NO: 16 | X | X | X | X | X | X | X | X |

It is to be understood that any one of the above combination of an enzyme being an enzyme exhibiting endo-1,4-beta-xylanase activity with an enzyme exhibiting endo-1,3(4)-beta-glucanase activity may be combined with a ratio between the two enzymes of 1:10, 2:10, 3:10, 4:10, 5:10, 6:10, 7:10, 8:10, 9:10, 10:10, 10:9, 10:8, 10:7, 10:6, 10:5, 10:4, 10:3, 10:2, or 10:1, such as within a range of 1:10-10:1, such as 2:10-10:2, such as 3:10-10:3, such as 4:10-10:4, such as 5:10-10:5, such as 6:10-10:6, such as 7:10-10:7, such as 8:10-10:8, or within 9:10-10:9. It is to be understood that any one of the above combination of an enzyme being an enzyme exhibiting endo-1,4-beta-xylanase activity with an enzyme exhibiting endo-1,3(4)-beta-glucanase activity may be combined with a ratio between the two enzymes of 1:1.

In some embodiments the composition according to the invention comprises a combination of at least two enzymes, said two enzymes, or two enzymes with an amino acid sequence having at least 80% sequence identity with the respective SEQ ID, or any functional fragment thereof, being selected from the list consisting of: SEQ ID NO 1 and SEQ ID NO:7; SEQ ID NO 2 and SEQ ID NO:7; SEQ ID NO 3 and SEQ ID NO:7; SEQ ID NO 4 and SEQ ID NO:7; SEQ ID NO 5 and SEQ ID NO:7; SEQ ID NO 6 and SEQ ID NO:7; SEQ ID NO 17 and SEQ ID NO:7; SEQ ID NO 18 and SEQ ID NO:7; SEQ ID NO 1 and SEQ ID NO:8; SEQ ID NO 2 and SEQ ID NO:8; SEQ ID NO 3 and SEQ ID NO:8; SEQ ID NO 4 and SEQ ID NO:8; SEQ ID NO 5 and SEQ ID NO:8; SEQ ID NO 6 and SEQ ID NO:8; SEQ ID NO 17 and SEQ ID NO:8; SEQ ID NO 18 and SEQ ID NO:8; SEQ ID NO 1 and SEQ ID NO:9; SEQ ID NO 2 and SEQ ID NO:9; SEQ ID NO 3 and SEQ ID NO:9; SEQ ID NO 4 and SEQ ID NO:9; SEQ ID NO 5 and SEQ ID NO:9; SEQ ID NO 6 and SEQ ID NO:9; SEQ ID NO 17 and SEQ ID NO:9; SEQ ID NO 18 and SEQ ID NO:9; SEQ ID NO 1 and SEQ ID NO:10; SEQ ID NO 2 and SEQ ID NO:10; SEQ ID NO 3 and SEQ ID NO:10; SEQ ID NO 4 and SEQ ID NO:10; SEQ ID NO 5 and SEQ ID NO:10; SEQ ID NO 6 and SEQ ID NO:10; SEQ ID NO 17 and SEQ ID NO:10; SEQ ID NO 18 and SEQ ID NO:10; SEQ ID NO 1 and SEQ ID NO:11; SEQ ID NO 2 and SEQ ID NO:11; SEQ ID NO:3 and SEQ ID NO:11; SEQ ID NO:4 and SEQ ID NO:11; SEQ ID NO:5 and SEQ ID NO:11; SEQ ID N0:6 and SEQ ID NO:11; SEQ ID N0:17 and SEQ ID NO:11; SEQ ID N0:18 and SEQ ID NO:11; SEQ ID NO:1 and SEQ ID NO:12; SEQ ID NO:2 and SEQ ID NO:12; SEQ ID NO:3 and SEQ ID NO:12; SEQ ID NO:4 and SEQ ID NO:12; SEQ ID NO:5 and SEQ ID NO:12; SEQ ID NO:6 and SEQ ID NO:12; SEQ ID NO:17 and SEQ ID NO:12; SEQ ID NO:18 and SEQ ID NO:12; SEQ ID NO:1 and SEQ ID NO:13; SEQ ID NO:2 and SEQ ID NO:13; SEQ ID NO:3 and SEQ ID NO:13; SEQ ID NO:4 and SEQ ID NO:13; SEQ ID NO: and SEQ ID NO: 13; SEQ ID NO:6 and SEQ ID NO:13; SEQ ID NO:17 and SEQ ID NO:13; SEQ ID NO:18 and SEQ ID NO:13; SEQ ID NO:1 and SEQ ID NO:14; SEQ ID NO:2 and SEQ ID NO:14; SEQ ID NO:3 and SEQ ID NO:14; SEQ ID NO:4 and SEQ ID NO:14; SEQ ID NO:5 and SEQ ID NO:14; SEQ ID NO:6 and SEQ ID NO:14; SEQ ID NO:17 and SEQ ID NO:14; SEQ ID NO:18 and SEQ ID NO:14; SEQ ID NO:1 and SEQ ID NO:15; SEQ ID NO:2 and SEQ ID NO:15; SEQ ID NO:3 and SEQ ID NO:15; SEQ ID NO:4 and SEQ ID NO:15; SEQ ID NO:5 and SEQ ID NO:15; SEQ ID NO:6 and SEQ ID NO:15; SEQ ID NO:17 and SEQ ID NO:15; SEQ ID NO:18 and SEQ ID NO:15; SEQ ID NO:1 and SEQ ID NO:16; SEQ ID NO:2 and SEQ ID NO:16; SEQ ID NO:3 and SEQ ID NO:16; SEQ ID NO:4 and SEQ ID NO:16; SEQ ID NO:5 and SEQ ID NO:16; SEQ ID NO:6 and SEQ ID NO:16; SEQ ID NO:17 and SEQ ID NO:16; and SEQ ID NO:18 and SEQ ID NO:16.

In some embodiments, the methods comprise an enzyme having xylanase activity of SEQ ID NO:1 and an enzyme exhibiting endo-1,3(4)-β-glucanase activity of SEQ ID NO:7. In some embodiments, the methods comprise an enzyme having xylanase activity of SEQ ID NO:2 and an enzyme exhibiting endo-1,3(4)-β-glucanase activity of SEQ ID NO:8. In some embodiments, the methods comprise an enzyme having xylanase activity of SEQ ID NO:2 and an enzyme exhibiting endo-1,3(4)-β-glucanase activity of SEQ ID NO:8. In some embodiments, any of the above combination of an enzyme being an enzyme exhibiting endo-1,4-beta-xylanase activity with an enzyme exhibiting endo-1,3(4)-beta-glucanase activity may be combined with a ratio between the two enzymes of 1:1 per weight. In some embodiments, any of the above combination, comprise an enzyme exhibiting endo-1,3(4)-beta-glucanase activity having a minimum activity of 9000 U/g and an enzyme exhibiting endo-1,4-beta-xylanase activity having a minimum activity of 13000U/g.

In some embodiments the composition and methods according to the invention comprises the use of any suitable commercially available enzymes having xylanase activity and/or beta-glucanase activity such as UltraFlo L (available from Novozymes-beta glucanase with cellulase, xylanase side activities), UltraFlo XL (available from Novozymes-beta glucanase with xylanase and alpha amylase side activities), UltraFlo Max (available from Novozymes-beta glucanase and xylanase), Finizyme 250 L(available from Novozymes-beta glucanase with cellulase, xylanase side activities) Filtrase series(available from DSM-beta-glucanase and xylanases).

In some embodiments the endo-1,3(4)-beta-glucanase activity and the endo-1,4-beta-xylanase activity are derived from at least two different enzymes. In some embodiments, the two different enzymes are from two different species.

In some embodiments the composition according to the invention comprises any one or more further enzyme. In some embodiments the one or more further enzyme is selected from a list consisting of a xylanase classified in EC 3.2.1.32, EC 3.2.1.136, or EC 3.2.1.156, a cellulase, a laminarinase, an endo-1,5-α-L-arabinanase, a beta-D-glucoside glucohydrolase, a beta -Xylosidase, a cellobiohydrolase, a glucan 1,4-beta-glucosidase, a xyloglucan-specific exobeta-1,4-glucanase and an alpha Arabinofuranosidase.

Sequences and enzymes identified by a sequence as mentioned herein and used according to the present invention alone or in combinations with other enzymes or compounds may be with or without signal peptide.

Methods

In one aspect, the present invention is directed to methods for the preparation of malted cereals comprising adding one or more enzymes to the malting process. In some embodiments, the present invention provides methods for the preparation of malted cereals comprising adding a cell wall degrading enzyme alone or in combination with other enzymes at some point during the malting process. In some embodiments, the cell wall degrading enzyme is a xylanase or a beta glucanase. In one embodiment, the present invention provides methods for the preparation of malted cereals comprising adding a beta glucanase alone or in combination with other enzymes at some point during the malting process. In one embodiment, the present invention provides compositions and methods for the preparation of malted cereals comprising adding a xylanase alone or in combination with other enzymes at some point during the malting process. In some embodiments, the other enzymes are other cell wall degrading enzymes. In some embodiments the invention provides compositions and methods for the preparation of malted cereals comprising adding a beta glucanase and a xylanase alone or in combination with other enzymes during the malting process.

In some embodiments, the present invention provides methods for the preparation process of malted cereals comprising the following steps: a steeping step including one or more wetting stages, a germination step, a drying step (e g kilning) including one or more temperature, and one or more enzymes as described herein which are added one or more times during the process. In some embodiments, the same or different one or more enzymes are added in one or more of the wetting stages of the steeping process. In some embodiments, the steeping step and/or the germination step involve spraying the cereal, and one or more enzymes as described herein are added one or more times during the process. In some embodiments, the one or more enzymes as described herein are added constantly during the spraying of the cereal.

In some embodiments, the cereals to be malted according to the methods described herein are selected from the group consisting of barley, wheat, rye, corn, oats, rice, millet, triticale, cassava, sorghum and a combination thereof.

The one or more enzymes described herein (e.g. a beta glucanase and/or a xylanase) may be introduced before or during the malting process. For example, the one or more enzymes described herein (e.g. a beta glucanase and/or a xylanase) may be introduced during the various malting stages before or after steeping of the cereal. In some embodiments the one or more enzymes described herein are added at the final stages of the malting process or after the malting process is completed to further increase the enzymatic activity of the malt.

In some embodiments, the preparation process of malted cereals comprises a steeping step including one or more wetting stages, where one or more enzymes as described herein are added one or more times during one or more of wetting stages. In some embodiments, the same or different enzymes are added one or more times during one or more of wetting stages of the steeping process. In some embodiments, the one or more wetting stages involve spraying the cereal, where one or more enzymes as described herein are added one or more times or constantly during the spraying.

The concentration of one or more enzymes described herein (e.g., a beta glucanase and/or a xylanase) may vary depending on the conditions of the malting process, the type of cereal (e.g., β-glucans and arabinoxylans content in the grain), the desired specification of the final malted product (e.g. viscosity, β-glucans and arabinoxylans content and final enzyme activity in the malt product, etc. . . . ) and the type of enzyme(s) being utilized. Generally about 0.01-100 mg enzyme protein per kilogram of cereals per gram air dry cereal is utilized. In some embodiments, about 0.1-50 mg/kg, or about 1-15 mg/kg or about 10-100 mg/kg are utilized. In some embodiments, about 3.75 mg/kg, or about 5 mg/kg, or about 6.25 mg/kg; or about 7.5 mg/kg; or about 10 mg/kg or about 12.5 mg/kg are utilized. In some embodiments, about 10 U/kg-1000 U/kg are utilized. In some embodiments, about 10 U/kg-100 U/kg, or about 30 U/kg-100 U/kg, or about 50 U/kg-100 U/kg are utilized. In some embodiments about 100 U/kg-1000 U/kg are utilized.

In some embodiments, the methods comprise an enzyme having xylanase activity selected from the group consisting of SEQ ID NO:1, SEQ ID NO:2, SEQ ID NO:3, SEQ ID NO:4,

SEQ ID NO:5, SEQ ID NO:6, SEQ ID NO:17, and SEQ ID NO:18 , or any functional fragment thereof. In some embodiments, the methods comprise an enzyme exhibiting endo-1,3(4)-β-glucanase activity selected from the group consisting of SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof. In some embodiments, the methods comprise an enzyme exhibiting endo-1,3(4)-beta-glucanase activity in combination with an enzyme exhibiting xylanase activity, each independently selected from the groups described above. In some embodiments, the methods comprise an enzyme having xylanase activity of SEQ ID NO:1 and an enzyme exhibiting endo-1,3(4)-β-glucanase activity of SEQ ID NO:7. In some embodiments, the methods comprise an enzyme having xylanase activity of SEQ ID NO:2 and an enzyme exhibiting endo-1,3(4)-β-glucanase activity of SEQ ID NO:8. In some embodiments, the methods comprise an enzyme having xylanase activity of SEQ ID NO:2 and an enzyme exhibiting endo-1,3(4)-β-glucanase activity of SEQ ID NO:8. In some embodiments, any of the above combination of an enzyme being an enzyme exhibiting endo-1,4-beta-xylanase activity with an enzyme exhibiting endo-1,3(4)-beta-glucanase activity may be combined with a ratio between the two enzymes of 1:1 per weight. In some embodiments, any of the above combination, comprise an enzyme exhibiting endo-1,3(4)-beta-glucanase activity having a minimum activity of 9000 U/g and an enzyme exhibiting endo-1,4-beta-xylanase activity having a minimum activity of 13000U/g.

In some embodiments the composition and methods according to the invention comprises the use of any suitable commercially available enzymes having xylanase activity and/or beta-glucanase activity such as UltraFlo L (available from Novozymes-beta glucanase with cellulase, xylanase side activities), UltraFlo XL (available from Novozymes-beta glucanase with xylanase and alpha amylase side activities), UltraFlo Max (available from Novozymes-beta glucanase and xylanase), Finizyme 250 L(available from Novozymes-beta glucanase with cellulase, xylanase side activities) Filtrase series(available from DSM-betaglucanase and xylanases).

In some embodiments, the cereal and one or more enzymes described herein (e.g., a beta glucanase and/or a xylanase) are combined at an effective concentration during one or more of the wetting of the cereal, and the combination is held at a temperature (e.g. of at least about 5° C. and not more than about 30° C., preferably between about 10° C. to about 20° C.) for a period of time to hydrolyse at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the β-glucans and/or arabinoxylans in grain. In some embodiments, the cereal and one or more enzymes described herein (e.g., a beta glucanase and/or a xylanase) are combined to hydrolyze at least 50% of the β-glucans and/or arabinoxylans in grain. In some embodiments, the cereal and one or more enzymes described herein (e.g., a beta glucanase and/or a xylanase) are combined to hydrolyze at least 60% of the β-glucans and/or arabinoxylans in grain. In some embodiments, the cereal and one or more enzymes described herein (e.g., a beta glucanase and/or a xylanase) are combined to hydrolyze at least 70% of the β-glucans and/or arabinoxylans in grain. In some embodiments, the cereal and one or more enzymes described herein (e.g., a beta glucanase and/or a xylanase) are combined to hydrolyze at least 80% of the β-glucans and/or arabinoxylans in grain.

In some embodiments, at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the β-glucans and/or arabinoxylans in grain are broken down compared to a control malted cereal prepared without the one or more enzymes according to the methods described herein. In some embodiments, at least 10% of the β-glucans and/or arabinoxylans in grain are broken down compared to a control malted cereal prepared without the one or more enzymes according to the methods described herein. In some embodiments, at least 50% of the β-glucans and/or arabinoxylans in grain are broken down compared to a control malted cereal prepared without the one or more enzymes according to the methods described herein. In some embodiments, at least 60% of the β-glucans and/or arabinoxylans in grain are broken down compared to a control malted cereal prepared without the one or more enzymes according to the methods described herein. In some embodiments, at least 70% of the β-glucans and/or arabinoxylans in grain are broken down compared to a control malted cereal prepared without the one or more enzymes according to the methods described herein. In some embodiments, at least 80% of the β-glucans and/or arabinoxylans in grain are broken down compared to a control malted cereal prepared without the one or more enzymes according to the methods described herein.

In some embodiments, at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the β-glucans in grain are broken down compared to a control malted cereal prepared without the one or more enzymes according to the methods described herein. In some embodiments, at least 10% of the β-glucans in grain are broken down compared to a control malted cereal prepared without the one or more enzymes according to the methods described herein. In some embodiments, at least 50% of the 13-glucans in grain are broken down compared to a control malted cereal prepared without the one or more enzymes according to the methods described herein. In some embodiments, at least 60% of the β-glucans in grain are broken down compared to a control malted cereal prepared without the one or more enzymes according to the methods described herein. In some embodiments, at least 80% of the β-glucans in grain are broken down compared to a control malted cereal prepared without the one or more enzymes according to the methods described herein.

In some embodiments, the β-glucans in grain are broken down such that the concentration of high molecular weight β-glucan in the malted cereal is less than 150 mg/l , or less than 100 mg/l or less than 90 mg/l , or less than 80 mg/l , or less than 70 mg/l , or less than 60 mg/l , or less than 50 mg/l. In some embodiments, the β-glucans in grain are broken down such that the concentration of high molecular weight β-glucan in the malted cereal is 50 mg/l or less.

In some embodiments, at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the arabinoxylans in grain are broken down compared to a control malted cereal prepared without the one or more enzymes according to the methods described herein. In some embodiments, at least 10% of the arabinoxylans in grain are broken down compared to a control malted cereal prepared without the one or more enzymes according to the methods described herein

In some embodiments, at least 50% of the β-glucans in grain are broken down and at least 50% of the arabinoxylans are broken down compared to a control malted cereal prepared without the one or more enzymes according to the methods described herein. In some embodiments, at least 60% of the β-glucans in grain are broken down and at least 50% of the arabinoxylans are broken down compared to a control malted cereal prepared without the one or more enzymes according to the methods described herein. In some embodiments, at least70% of the β-glucans in grain are broken down and at least 50% of the arabinoxylans are broken down compared to a control malted cereal prepared without the one or more enzymes according to the methods described herein. In some embodiments, at least 80% of the β-glucans in grain are broken down and at least 50% of the arabinoxylans are broken down compared to a control malted cereal prepared without the one or more enzymes according to the methods described herein.

In some embodiments, the arabinoxylans in grain are broken down such that the concentration of high molecular weight arabinoxylans in the malted cereal is less than 2400 mg/l , or less than 2200 mg/l , or less than 1900 mg/l , or less 1500 than mg/l , or less than 1000 mg/l , or less than 800 mg/l , or less than 700 mg/l or less than 600 mg/l , or less than 500 mg/l , or less than 100 mg/l , or less than 60 mg/l , or less than 50 mg/l. In some embodiments, the arabinoxylans in grain are broken down such that the concentration of high molecular weight arabinoxylans in the malted cereal is 2000 mg/l or less.

In some embodiments, the cereal and one or more enzymes described herein (e.g., a beta glucanase and/or a xylanase) are combined at an effective concentration during one or more of the wetting of the cereal at a specific temperature(s) for a period of time until the cereal has a moisture content of at least about 20 weight percent. In yet other embodiments, after the wetted cereal has attained increased moisture content and has started to germinate. In still other embodiments, the moistened cereal and the one or more enzymes are combined at an effective concentration during one or more of the wetting of the cereal for a period of time and temperature until the cereal has a moisture content of between about 20 to about 60 weight percent, or about 40 to 50 weight percent. In some embodiments the moistened cereal and the one or more enzymes are combined at an effective concentration during one or more of the wetting of the cereal for a period of time and temperature until the cereal has a moisture content of between about 40 to about 47 weight percent, and has germinated. In some embodiments, the total time of the steeping process is less compared to the time of a control malted cereal prepared without the one or more enzymes according to the methods described herein.

In still other embodiments, the moistened cereal and the one or more enzymes are combined at an effective concentration during one or more of the wetting of the cereal for a period of time and temperature until the cereal has a moisture content of between about 20 to about 60 weight percent, or about 40 to 50 weight percent, and the β-glucans in grain are broken down such that the concentration of high molecular weight β-glucan in the malted cereal is 50 mg/l or less. In some embodiments the moistened cereal and the one or more enzymes are combined at an effective concentration during one or more of the wetting of the cereal for a period of time and temperature until the cereal has a moisture content of between about 40 to about 47 weight percent, has germinated and the β-glucans in grain are broken down such that the concentration of high molecular weight β-glucan in the malted cereal is 50 mg/l or less. In some embodiments, the total time of the steeping process is less compared to the time of a control malted cereal prepared without the one or more enzymes according to the methods described herein.

In still other embodiments, the moistened cereal and the one or more enzymes are combined at an effective concentration during one or more of the wetting of the cereal for a period of time and temperature until the cereal has a moisture content of between about 20 to about 60 weight percent, or about 40 to 50 weight percent, and the arabinoxylans in grain are broken down such that the concentration of high molecular weight arabinoxylans in the malted cereal is 2000 mg/l or less. In some embodiments the moistened cereal and the one or more enzymes are combined at an effective concentration during one or more of the wetting of the cereal for a period of time and temperature until the cereal has a moisture content of between about 40 to about 47 weight percent, has germinated and the arabinoxylans in grain are broken down such that the concentration of high molecular weight arabinoxylans in the malted cereal is 2000 mg/l or less. In some embodiments the moistened cereal and the one or more enzymes are combined at an effective concentration during one or more of the wetting of the cereal for a period of time and temperature until the cereal has a moisture content of between about 40 to about 47 weight percent, has germinated and the arabinoxylans in grain are broken down such that the concentration of high molecular weight arabinoxylans in the malted cereal is 1000 mg/l or less. In some embodiments, the total time of the steeping process is less compared to the time of a control malted cereal prepared without the one or more enzymes according to the methods described herein.

In some embodiments, the total time for the steeping process is at least 5, 7, 9, 10, 11, 13, 15, 17, 19, 20, 21, 23, 25, 27, 29, 30, 31, 33, 35, 37, 39, 40, 41, 43, 45, 47, 49, 50, 51, 53, 55, 57, 59, 60, 61, 63, 65, 67, 69, 70, 71, 73, 75, 77, 79, 80, 81, 83, 85, 87, 89, 91, 93 or 95% less than the time of a control malted cereal prepared without the one or more enzymes according to the methods described herein.

In some embodiments, the total time for the steeping process does not exceed 30 hours. In some embodiments, the total time for the steeping process does not exceed 25 hours. In some embodiments, the total time for the steeping process is about 10 hours to about 25 hours. In some embodiments, the total time for the steeping process is less than 25, 20, 15, or even less than 10 hours.

In some embodiments, the total amount of water used during the steeping process is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% less than the water used for a control malted cereal prepared without the one or more enzymes according to the methods described herein. In some embodiments, the total amount of water used during the steeping process is at least 10% less than the water used for a control malted cereal prepared without the one or more enzymes according to the methods described herein. In some embodiments, the total amount of water used during the steeping process is at least 20% less than the water used for a control malted cereal prepared without the one or more enzymes according to the methods described herein. In some embodiments, the total amount of water used during the steeping process is at least 30% less than the water used for a control malted cereal prepared without the one or more enzymes according to the methods described herein.

In some embodiments, the invention provides for improved water uptake by the grain. In some embodiments, the water uptake by the grain is improved by at least 10, 15, 20, 50, 70, 90 or 95% compared to a control malted cereal prepared without the one or more enzymes according to the methods described herein. In some embodiments, the water uptake by the grain is improved by at least 10%, or at least 20%.

In some embodiments, the cereal and one or more enzymes described herein (e.g., a beta glucanase and/or a xylanase) are combined at an effective concentration at the beginning of the first wetting of the cereal. In some embodiments, the cereal and one or more enzymes described herein (e.g., a beta glucanase and/or a xylanase) are combined at an effective concentration during the first wetting of the cereal. In some embodiments, the cereal and one or more enzymes described herein (e.g., a beta glucanase and/or a xylanase) are combined at an effective concentration during the first wetting of the cereal after a desired temperature have been reached. In some embodiments, the wetting stage involves spraying the cereal, where one or more enzymes as described herein are added one or more times, or constantly, during the spraying.

In some embodiments, the cereal and one or more enzymes described herein (e.g., a beta glucanase and/or a xylanase) are combined at an effective concentration 1, 2, 3 or 4 hours after the beginning of the first wetting of the cereal. In some embodiments, the cereal and one or more enzymes described herein (e.g., a beta glucanase and/or a xylanase) are combined during the last hour of the first wetting of the cereal.

In some embodiments, the cereal and one or more enzymes described herein (e.g., a beta glucanase and/or a xylanase) are combined at an effective concentration at the beginning of the second wetting of the cereal. In some embodiments, the cereal and one or more enzymes described herein (e.g., a beta glucanase and/or a xylanase) are combined at an effective concentration during the second wetting of the cereal. In some embodiments, the cereal and one or more enzymes described herein (e.g., a beta glucanase and/or a xylanase) are combined at an effective concentration during the second wetting of the cereal after a desired temperature have been reached. In some embodiments, the wetting stage involves spraying the cereal, where one or more enzymes as described herein are added one or more times, or constantly, during the spraying.

In some embodiments, the cereal and one or more enzymes described herein (e.g., a beta glucanase and/or a xylanase) are combined at an effective concentration 1, 2, 3 or 4 hours after the beginning of the second wetting of the cereal. In some embodiments, the cereal and one or more enzymes described herein (e.g., a beta glucanase and/or a xylanase) are combined during the last hour of the second wetting of the cereal.

In some embodiments, the cereal and one or more enzymes described herein (e.g., a beta glucanase and/or a xylanase) are combined at an effective concentration at the beginning of the third wetting of the cereal. In some embodiments, the cereal and one or more enzymes described herein (e.g., a beta glucanase and/or a xylanase) are combined at an effective concentration during the third wetting of the cereal. In some embodiments, the cereal and one or more enzymes described herein (e.g., a beta glucanase and/or a xylanase) are combined at an effective concentration during the third wetting of the cereal after a desired temperature have been reached. In some embodiments, the wetting stage involves spraying the cereal, where one or more enzymes as described herein are added one or more times, or constantly, during the spraying.

In some embodiments, the cereal and one or more enzymes described herein (e.g., a beta glucanase and/or a xylanase) are combined at an effective concentration 1, 2, 3 or 4 hours after the beginning of the third wetting of the cereal. In some embodiments, the cereal and one or more enzymes described herein (e.g., a beta glucanase and/or a xylanase) are combined during the last hour of the third wetting of the cereal.

In some embodiments, the cereal and one or more enzymes described herein (e.g., a beta glucanase and/or a xylanase) are combined at an effective concentration at the beginning of each of the wetting stages of the cereal. In some embodiments, the cereal and one or more enzymes described herein (e.g., a beta glucanase and/or a xylanase) are combined at an effective concentration during all the wetting stages of the cereal. In some embodiments, the cereal and one or more enzymes described herein (e.g., a beta glucanase and/or a xylanase) are combined at an effective concentration during all the wetting stages of the cereal after a desired temperature have been reached. In some embodiments, the one or more wetting stages involve spraying the cereal, where one or more enzymes as described herein are added one or more times, or constantly, during the spraying.

In some embodiments, the present invention provides malted cereals, obtained according to the process of the invention, which present improved properties. For instance, the malted cereals produced by the methods described herein have a lower high molecular weight fraction of beta-glucan and/or arabinoxylans content, and/or a higher enzyme activity such as, for example, beta-glucanase or xylanase activity than a control malted cereal prepared without the one or more enzymes according to the methods described herein. This allows for a better processability of the malt, e.g., in wort and beer production by potentially having increased rates of mash separation and beer filtration. In some embodiments, the malted cereals produced by the methods described herein have a concentration of high molecular weight β-glucan of 100 mg/l, 90 mg/l, 80 mg/l, 70 mg/l, 60 mg/l, 50 mg/l or less. In some embodiments, the malted cereals produced by the methods described herein have a concentration of high molecular weight arabinoxylans of 2500 mg/l, 2200 mg/l, 2100 mg/l, 1900 mg/l, 1500 mg/l, 1000 mg/1800 mg/l, 700 mg/l, 600 mg/l, 500 mg/l, 100 mg/l, 50 mg/l or less. In some embodiments, malted cereals produced by the methods described herein have a beta-glucanase activity and/or xylanase activity of about 10 U/kg-100 U/kg, or about 30 U/kg-100 U/kg, or about 50 U/kg-100 U/kg are utilized. In some embodiments about 100 U/kg-1000 U/kg.

In some embodiments, an object of the present invention concerns the use of the malted cereals according to the invention for the preparation of food and beverages. The invention is also related to these improved beverages. The improved malted cereals according to the invention could also be used in other biotechnological processes well known by a person of ordinary skill in the art, in which in most cases advantage is taken of their improved quality.

In some embodiments, the methods describes herein provide for a malted cereal with a decrease amount of high molecular weight fraction of β-glucans and/or arabinoxylans. In some embodiments, the total amount of high molecular weight β-glucans and/or arabinoxylans is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% less than the amount of high molecular weight β-glucans and/or arabinoxylans in a control malted cereal prepared without the one or more enzymes according to the methods described herein.

In some embodiments, the total amount of high molecular weight β-glucans is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% less than the amount of β-glucans in a control malted cereal prepared without the one or more enzymes according to the methods described herein. In some embodiments, the total amount of high molecular weight β-glucans is at least 50% less than the amount of high molecular weight β-glucans in a control malted cereal prepared without the one or more enzymes according to the methods described herein. In some embodiments, the total amount of high molecular weight β-glucans is at least 60% less than the amount of high molecular weight β-glucans in a control malted cereal prepared without the one or more enzymes according to the methods described herein. In some embodiments, the total amount of high molecular weight β-glucans is at least 70% less than the amount of high molecular weight β-glucans in a control malted cereal prepared without the one or more enzymes according to the methods described herein. In some embodiments, the total amount of high molecular weight β-glucans is at least 80% less than the amount of high molecular weight β-glucans in a control malted cereal prepared without the one or more enzymes according to the methods described herein.

In some embodiments, the total amount of arabinoxylans is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% less than the amount of arabinoxylans in a control malted cereal prepared without the one or more enzymes according to the methods described herein. In some embodiments, the total amount of high molecular weight arabinoxylans is at least 50% less than the amount of high molecular weight arabinoxylans in a control malted cereal prepared without the one or more enzymes according to the methods described herein.

In some embodiments, the total amount of high molecular weight arabinoxylans is at least 50% less than the amount of high molecular weight arabinoxylans and the total amount of high molecular weight β-glucans is at least 80% less than in a control malted cereal prepared without the one or more enzymes according to the methods described herein.

In some embodiments, the methods described herein provide for a malted cereal with lower viscosity, when extracted. In some embodiments, the viscosity of the malted cereal is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% lower than the viscosity of a malted cereal prepared without the one or more enzymes according to the invention.

In some embodiments, the malted cereals described herein provide for the production of an extract with lower viscosity. In some embodiments, the viscosity of the extract is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% lower than the viscosity of a extract prepared with a control malted cereal prepared without the one or more enzymes according to the invention.

In some embodiments, the process of the invention may increase the enzymatic activity of the malted cereal. In some embodiments, the enzymatic activity is selected from the group consisting ofβ-glucanase activity, xylanase activity, amylase activity, protease activity, naturally occurring enzyme activity in the cereal and combinations thereof.

In some embodiments, the process of the invention may increase aβ-glucanase activity and/or a xylanase activity of a malted cereal by a factor of at least about 2 as compared to a control malted cereal prepared without the one or more enzymes according to the invention.

In some embodiments, a β-glucanase activity and/or a xylanase activity of a malted cereal is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% higher than a control malted cereal prepared without the one or more enzymes according to the invention. In some embodiments, malted cereals produced by the methods described herein have a beta-glucanase activity and/or xylanase activity of about 10 U/kg-100 U/kg, or about 30 U/kg-100 U/kg, or about 50 U/kg-100 U/kg are utilized. In some embodiments about 100 U/kg-1000 U/kg.

Thus is some embodiments, the cereal and one or more enzymes described herein (e.g., a beta glucanase and/or a xylanase) are combined at an effective concentration during at least one of the wetting of the cereal for a period of time and at a temperature to obtain an increase in enzymatic activity of the malted cereal. In some embodiments, the enzymatic activity is selected from the group consisting of β-glucanase activity, xylanase activity, amylase activity, protease activity, naturally occurring enzyme activity in the cereal and combinations thereof.

In some embodiments the one or more enzymes described herein are added at the final stages of the malting process or after the malting process is completed to further increase the enzymatic activity of the malt.

In some embodiments, the methods described herein provide for a malted cereal with a lower potential for off flavor formation, such as off flavor formation related to arabinoxylan breakdown.

In some embodiments the methods described herein provide for a malted cereal with a decreased risk of filter bed collapse, such as at lautering.

In some embodiments the total pressure built up is reduced to a value of less than 470 mm WC, such as less than 450 mm WC, such as less than 430 mm WC, such as less than 410 mm WC, such as less than 390 mm WC, such as less than 370 mm WC, such as less than 350 mm WC, such as less than 330 mm WC, such as less than 310 mm WC, such as less than 300 mm WC, such as less than 290 mm WC, when a malted cereal according to the present invention is used prior to the mash separation in a brewing application.

In some embodiments the total pressure built up is reduced by at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% compared to the use of a negative control without said malted cereal; when used prior to the mash separation in a brewing application.

In some embodiments the mash separation as measured by volume wort collected after 5 min of separation relative to a control malted cereal is increased to above 1.5, such as above 1.6, such as above 1.7, such as above 1.8, such as above 1.9, such as above 2.0, such as above 2.1, such as above 2.2, such as above 2.3, such as above 2.4, such as above 2.5, when the composition according to the invention is used in a brewing application prior to the mash separation.

In some embodiments the mash separation as measured by volume wort collected after 5 min of filtration is increased at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 110, 120, 130, 140, 150, 160, 170, 180, 190, 200, 210, 220, 230, 240, 250, 260, 270, 280, 290, or 300% as compared to the use of a control malted cereal.

The process of malting is well known in the art and the specific conditions vary depending on the type of cereal used, the final specifications needed for the malted cereals and the specific malting facilities. The methods described herein can be applied to any malting process known in the art and can be used in any malting facility. It is to be understood that certain parameters of the compositions and methods described herein may be adjusted depending to the properties needed for the malt and/or the specific malting facilities to obtain such optimal properties in the malted cereal, e.g., depending on the cereal varieties and the downstream uses.

### EXAMPLES

The present disclosure is described in further detail in the following examples, which are not in any way intended to limit the scope of the disclosure as claimed. The following examples are offered to illustrate, but not to limit the claimed disclosure.

### Example 1

### Assays to Measure Enzyme Activity

DNS Cellulase activity method (DNS CMC method)

Systematic Name: 1,4-(1,3;1,4)- beta-D-glucan 4-glucanohydrolase IUB Number: EC 3.2.1.4

Principle

The assay of cellulase is based on the enzymatic endo-hydrolysis of the 1,4-β-D-glucosidic bonds in carboxymethylcellulose (CMC), a 13-1,4-glucan. The products of the reaction (β-1,4 glucan oligosaccharides) is determined colorimetrically by measuring the resulting increase in reducing groups using a 3,5 dinitrosalicylic acid reagent. Enzyme activity is calculated from the relationship between the concentration of reducing groups, as glucose equivalents, and absorbance at 540 nm.

The assay is carried out at pH 5.0, but it can be performed at different pH values for the additional characterization and specification of enzymes.

Unit definition

One unit of cellulase activity is defined as the amount of enzyme which produces 1 μmole glucose equivalents per minute under the conditions of the assay (pH 5.0 (or as specified) and 50° C.).

Materials

Carboxymethylcellulose. Supplier: Megazyme Ltd. Product no.: CM-Cellulose 4M; D-Glucose ‘AnalaR’. Supplier: Merck Ltd (BDH). Product no.: 10117. M.W.: 180.16; Sodium acetate anhydrous ‘AnalaR’. Supplier: Merck Ltd (BDH). Product no.: 10236. M.W.: 82.03; Acetic acid (“glacial”) ‘AnalaR’. Supplier: Merck Ltd (BDH). Product no.: 10001. M.W.: 60.05; 3,5-Dinitrosalicylic acid GPR (3,5-dinitro-2-hydroxybenzoic acid). Supplier: Merck Ltd (BDH). Product no.: 28235; Sodium hydroxide pellets ‘AnalaR’. Supplier: Merck Ltd

(BDH). Product no.: 10252. M.W.: 40.00; Potassium sodium (+)-tartrate ‘AnalaR’. Supplier: Merck Ltd (BDH). Product no.: 10219. M.W.: 282.22; 1.5%(w/v solution)

Carboxymethylcellulose (CMC) solution in 0.1M sodium acetate buffer, pH 5.0 (substrate solution); 3,5-Dinitrosalicylic acid (DNS) solution. 20g/L of DNS in buffer containing 32g/L sodium hydroxide pellets, and 600 g/L potassium sodium (+)-tartrate; Glucose standard solution (0.50 mg/ml)

Procedure

The enzyme composition is diluted into samples and a glucose standard curve can be made using glucose concentrations of 0, 0.125, 0.25, 0.375, and 0.5 mg/ml.

0.25 ml of enzyme solution is mixed with 1.75 ml of the substrate solution (1.5% w/v) at 50 ° C. and the reaction is stopped after 10 min by addition of DNS solution. This is followed by heating to 95° C. for 5 minutes.

The optical density is measured at 540 nm (OD540 nm) of the different samples.

Calculation

The enzyme activity is determined from the standard curve.

The activity is calculated as described in PCT publication number WO2013/037933.

Laminarinase (DNS laminarin method)

Principle

The reaction, catalysed by laminarinase, involves the endohydrolysis of 1,3-glucosidic bonds in 1,3-β-D-glucans. Substrates include laminarin, paramylon and pachyman. The products of the reaction (β3-glucan oligosaccharides) are determined colourimetrically by measuring the resulting increase in reducing groups using a 3,5 dinitrosalicylic acid reagent. Enzyme activity is calculated from the relationship between the concentration of reducing groups, as glucose equivalents, and absorbance at 540 nm.

The assay can be carried out at pH 5.0 and 50° C., it can also be performed at different values of pH and temperature for the additional characterization and specification of enzymes.

Unit Definition

One unit of laminarinase activity is defined as the amount of enzyme which produces 1 μmole glucose equivalents per minute under the conditions of the assay (pH 5.0 and 50° C. (or as specified)).

Materials

See materials given above for the Cellulase activity assay.

Laminarin (from Laminaria digitata). Supplier: Sigma-Aldrich Co. Ltd. Product no.: L 9634; 1.00%(w/v solution) Laminarin solution (substrate solution 0.1M sodium acetate buffer, pH 5.0); 1.75 ml laminarin solution is mixed with 0.25 ml diluted enzyme solution at 50° C. for 10 minutes and the reaction stopped by addition of 2 ml DNS solution.

Standard curve can be made using 0, 0.125, 0.25, 0.5 and 0.75 mg/ml glucose solution. Optical density is measured at 540 nm (OD540 nm).

Calculation

The activity is calculated as described in PCT publication number WO2013/037933.

Arabinase Assay

Principle

The assay of Arabinase activity is based on colorimetrically determination by measuring the resulting increase in reducing groups using a 3,5 dinitrosalicylic acid reagent. Enzyme activity is calculated from the relationship between the concentration of reducing groups, as arabinose equivalents, and absorbance at 540 nm.

The assay can be carried out at pH 3.5, it can also be performed at different pH values for the additional characterization and specification of enzymes.

Unit Definition

One unit of arabinase (Arabinanase (endo-1,5-alphα-L-arabinanase)) activity is defined as the amount of enzyme which produces 1 μmole arabinose equivalents per minute under the conditions of the assay (pH 3.5 (or as specified) and 50° C.).

Materials

Megazyme Sugar Beet Arabinan; Arabinose Sigma A3131 M.W.: 150.1;Sodium acetate anhydrous ‘AnalaR’. Supplier: Merck Ltd (BDH). Product no.: 10236. M.W.: 82.03; Acetic acid (“glacial”) ‘AnalaR’. Supplier: Merck Ltd (BDH). Product no.: 10001. M.W.: 60.05; 3,5-Dinitrosalicylic acid GPR (3,5-dinitro-2-hydroxybenzoic acid). Supplier: Merck Ltd (BDH). Product no.: 28235; Sodium hydroxide pellets ‘AnalaR’. Supplier: Merck Ltd (BDH). Product no.: 10252. M.W.: 40.00; Potassium sodium (+)-tartrate ‘AnalaR’. Supplier: Merck Ltd (BDH). Product no.: 10219. M.W.: 282.22; 1.5%(w/v solution) Arabinan solution in 0.1M sodium acetate buffer, pH 3.5 (substrate solution); 3,5-Dinitrosalicylic acid (DNS) solution. 20g/L of DNS in buffer containing 32g/L sodium hydroxide pellets, and 600 g/L potassium sodium (+)-tartrate; Arabinase standard solution (0.50 mg/ml)

Procedure

The enzyme composition is diluted into samples and an arabinase standard curve is made using arabinase concentrations of 0, 0.125, 0.25, 0.375, and 0.5 mg/ml.

0.25 ml of enzyme solution is mixed with 1.75 ml of the substrate solution (1.5% w/v) at 50 ° C. and the reaction is stopped after 10 min by addition of DNS solution. This is then followed by heating to 95° C. for 5 minutes.

The optical density is measured at 540 nm (OD540 nm) of the different samples.

Calculation

The enzyme activity is determined from the standard curve.

The activity is calculated as described in PCT publication number WO2013/037933.

Xylanase Activity Assay

Samples, to obtain approx. OD540 =0.25-0.30 in this assay and xylose standards (0, 0.125, 0.250, 0.375 and 0.500 mg/ml distilled water) are prepared in distilled water. At time t=0 minutes, 1.75 ml soluble wheat arabinoxylan (0.5% wheat arabinoxylan (PWAXYH, Megazyme, Bray, Ireland)) in 0.1M sodium acetate/acetic acid, pH 5) is placed in a test tube at 50° C. At time t=5 minutes, 250μ1 enzyme solution is added to the substrate at 50° C. followed by mixing. Distilled water is used as blank. At time t=15 minutes, 2m1 DNS solution (1% 3,5-Dinitrosalicylic acid (DNS), 1.6% sodium hydroxide, 30% potassium sodium tartrate in distilled water) is added to the enzyme-substrate solution and 2.0 ml standard solution. Samples, blanks and standards added DNS are placed in a boiling water bath (95° C.) for 5 minutes. Hereafter samples, blanks and standards are cooled by placing them in a 25° C. water bath for 20 minutes. The optical density of all samples are read at OD540 using a spectrophotometer. Based on the dilution of the samples, the amount of sample used and the standards, the xylanase activity of the sample can be calculated.

One Unit of endo-1,4-beta-xylanase activity is defined as the amount of enzyme which produces 1 μmole xylose equivalents per minute under the conditions mentioned above

Glucanase Activity Assay

Samples, to obtain OD540 within the standard curve in this assay and glucose standards (0; 0.125; 0.250; 0.500; and 0.750 mg/ml distilled water) are prepared in distilled water. At time t=0 minutes, 1,75 ml barley beta-glucan (1.5% barley beta-glucan (BETA-BGBM, Megazyme, Bray, Ireland)) in 1M sodium acetate/acetic acid, pH 5) is placed in a test tube at 50° C. At time t=5 minutes, 250μ1 enzyme solution is added to the substrate at 50° C. followed by mixing. Distilled water is used as blank. At time t=15 minutes, 2m1 DNS solution (1% 3,5-Dinitrosalicylic acid (DNS), 1,6% sodium hydroxide, 30% potassium sodium tartrate in distilled water) is added to the enzyme-substrate solution and 2.0 ml standard solution. Samples, blanks and standards added DNS are placed in a boiling water bath (95° C.) for 15 minutes. Hereafter samples, blanks and standards are cooled by placing them in a 25° C. water bath for 20 minutes. The Optical density of all samples are read at OD540 using a spectrophotometer. Based on the dilution of the samples, the amount of sample used and the standards, the glucanase activity of the sample can be calculated.

One unit of endo-1,3(4)-β-glucanase activity is defined as the amount of enzyme which produces 1 μmole glucose equivalents per minute under the conditions of the assay (pH 5.0 (or as specified) and 50° C.).

Arabinofuranosidase Assay

The reaction, catalysed by a-N-arabinofuranosidase, involves the hydrolysis of the terminal bond, at the non-reducing α-L-arabinofuranoside residue, of α-L-arabinosides. The enzyme acts on α-L-arabinofuranosides, α-L-arabinans containing (1,3)- and/or (1,5)-linkages, arabinoxylans and arabinogalactans.

The assay of a-N-arabinofuranosidase is based upon the enzymatic hydrolysis of p-nitrophenyl α-L-arabinofuranoside. The assay is a “two-point”, rather than a “continuous monitoring”, method. The calculation of enzyme activity is based on measurements taken only at the beginning and end of the incubation period. A product of the reaction, p-nitrophenol is determined colourimetrically (after pH adjustment). Enzyme activity is calculated from the relationship between the concentration of p-nitrophenol and absorbance at 400 nm.

Preparation of Diluted Enzyme Solution and Procedure

The enzyme solution is prepared and the assay is performed as described in PCT publication number WO2013/037933.

Calculation

The activity is calculated as described in PCT publication number WO2013/037933.

Cellobiohydrolase Assay

Principle

The reaction, catalyzed by cellobiohydrolase, involves the hydrolysis of 1,4-β-D-glucosidic linkages in cellulose and cellotetraose, releasing cellobiose from the non-reducing ends of the chains.

The assay of cellobiohydrolase is based on the enzymatic hydrolysis of p-nitrophenyl β-D-cellobiopyranoside. The product of the reaction, p-nitrophenol is determined colorimetrically (after pH adjustment). Enzyme activity is calculated from the relationship between the concentration of p-nitrophenol and absorbance at 400 nm.

The assay is operated within the linear defined range of αOD540 nm TEST (T)=0.400-0.800.

Procedure

The assay is performed as described in PCT publication number WO2013/037933.

Calculation

The activity is calculated as described in PCT publication number WO2013/037933.

### Example 2

A Joe White micro-malting system can be used to test multiple samples of different cereals from different varieties (e.g., barley: Sebastian, Tipple, Prestige, and Bellini), using different enzymes concentrations at different time points. This micro-malting system allows for steeping, germination, and kilning of samples in one unit while providing uniformity of malting conditions for each batch. Each sample is placed in one compartment of the micro-malting system. A sample of 0.5 kilogram of barley is added to each of the compartments in the micro-malting unit for malting. A mixture of enzymes containing an endo-1,3(4)-beta-glucanase and an endo-1,4-beta-xylanase is added at different times during the steeping process at the different concentrations 0.01-100 mg enzyme protein per kilogram. The table below describes experimental conditions.

| Compartment/ | Barley | |
|---|---|---|
| Sample | Variety | Experimental Conditions |
| 1 | Sebastian | No Enzymes |
| 2 | Sebastian | Enzymes added during the first |
| | | wet stage |
| 3 | Sebastian | Enzymes added during the first |
| | | and second wet stages |
| 4 | Sebastian | Enzymes added during all wet |
| | | stages |
| 5 | Tipple | No Enzymes |
| 6 | Tipple | Enzymes added during the first |
| | | wet stage |
| 7 | Tipple | Enzymes added during the first |
| | | and second wet stages |
| 8 | Tipple | Enzymes added during all wet |
| | | stages |
| 9 | Prestige | No Enzymes |
| 10 | Prestige | Enzymes added during the first |
| | | wet stage |
| 11 | Prestige | Enzymes added during the first |
| | | and second wet stages |
| 12 | Prestige | Enzymes added during all wet |
| | | stages |
| 13 | Bellini | No Enzymes |
| 14 | Bellini | Enzymes added during the first |
| | | wet stage |
| 15 | Bellini | Enzymes added during the first |
| | | and second wet stages |
| 16 | Bellini | Enzymes added during all wet |
| | | stages |

Three steeping cycles are used for all barley samples.

The barley can be steeped using different experimental conditions:

(a) the barley is steeped with the first water immersion for the time necessary for the grain to reach 40-45% moisture at a temperature of 15° C. Following the water immersion, the barley is subjected to air ventilation for 12 hours at a temperature of 15° C. The second immersion is for the time necessary for the grain to reach 40-45% moisture, which is then followed by air ventilation for 10 hours at 15° C. The third steeping is for 2 hours at 15° C. After steeping is completed, germination begins and the grains can be allowed to germinate for 5 days. The time necessary for each of the varieties to reach the desired levels of moisture is recorded.

(b) the barley is steeped with the first water immersion for 6 hours at a temperature of 15° C. with a total water to air dry barley ratio of 3:1 for samples with no enzymes, 2:1 for samples in which the enzymes are added during the first wet stage, 1:1 for samples in which enzymes are added during the first and second wet stages; and 0.8:1 for samples in which enzymes are added during all wet stages. Following the water immersion, the barley is subjected to air ventilation for 12 hours at a temperature of 15° C. The second immersion is for 5 hours, followed by air ventilation for 0 hours at 15° C. The third immersion is for 2 hours at 15° C. After steeping is completed, the varieties can be germinated for 5 days.

Conditions (a) and (b) can be repeated using different enzymes concentrations, to determine differences in water consumption and/or germination time.

The germinating barley is subjected to humidified air at a temperature of 15° C.

After 5 days of germination, the barley in each compartment is kilned with a standard kilning program. Malt yield is measured in 1,000 kernel weight for each sample from each of the 16 compartments.

The amount of beta-glucans and arabinoxylans present in the germinating barley and final malt can be measured by any suitable methods known in the art. For example the amount of beta-glucans present in the germinating barley and final malt can be measured by the methods described in Journal of the Institute of Brewing Volume 91, Issue 5, pages 285-295, September-October 1985.

The glucanase and/or xylanase activity present in the malted cereal can be measured as described above.

Six quality parameters are measured on each malt sample, i.e. extract yield (%), total malt protein (%), soluble malt protein (%), Kolbach Index, apparent final attenuation (%) and viscosity (cp). The analytical procedures are used according to those recommended by the European Brewery Convention.

### Example 3

Micromalting and Malting Quality Analysis

Micromalting and subsequent analyses can be performed with two-rowed spring barley Troubadour from different locations. Troubadour, bred in The Netherlands and released in Spain in 1983, is very high-yielding under Mediterranean environments. However, its malting quality is fairly poor, giving low extract yields. It has been reported that the low yield could be correlated with the content of beta-glucan in the grain.

A micromalting plant that allows the processing of 32 samples of 200 g each per batch can be used. The micromalting procedure is as follows:
Steeping time: 57 h (41 h under water and 16 h without water)
Steeping temperature: 15° C.
Germination time: 5 days
Germination temperature: 15° C.
Air supply to germinating barley: 50 ml/min
Drying time: 17 h
Drying temperature: 50° C.
Kilning time: 2.5 h
Kilning temperature: 70° C.

Steeping is performed in the presence or absence of a mixture of enzymes containing an endo-1,3(4)-beta-glucanase and an endo-1,4-beta-xylanase at different times during the steeping process and at the different concentrations ranging from 0.01-100 mg enzyme protein per kilogram.

Six quality parameters are measured on each malt sample, i.e. extract yield (%), total malt protein (%), soluble malt protein (%), Kolbach Index, apparent final attenuation (%) and viscosity (cp). The analytical procedures are used according to those recommended by the European Brewery Convention.

Additionally, the total protein content, the beta-glucan and arabinoxylan content of barley and the beta-glucanase and xylanase activity of malt are determined as described above.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. Sequences:

AtuXyn3, Aspergillus tubigensis (SEQ ID NO: 1),
302 aa:
QASVSIDTKFKAHGKKYLGNIGDQYTLTKNSKTPAIIKADFGALTPEN
SMKWDATEPSRGQFSFSGSDYLVNFAQSNNKLIRGHTLVWHSQLPSWV
QAITDKNTLIEVMKNHITTVMQHYKGKIYAWDVVNEIFNEDGSLRDSV
FYQVIGEDYVRIAFETARAADPNAKLYINDYNLDSASYPKLTGMVSHV
KKWIEAGIPIDGIGSQTHLSAGGGAGISGALNALAGAGTKEIAVTELD
IAGASSTDYVEVVEACLDQPKCIGITVWGVADPDSWRSSSTPLLFDSN
YNPKPAYTAIANAL
TerXyn1, Geosmithia emersonii (Taleromyces
emersonii) (SEQ ID NO: 2):
AGLNTAAKAIGLKYFGTATDNPELSDTAYETQLNNTQDFGQLTPANSM
KWDATEPEQNVFTFSAGDQIANLAKANGQMLRCHNLVWYNQLPSWVTS
GSWTNETLLAAMKNHITNVVTHYKGQCYAWDVVNEALNDDGTYRSNVF
YQYIGEAYIPIAFATAAAADPNAKLYYNDYNIEYPGAKATAAQNLVKL
VQSYGARIDGVGLQSHFIVGETPSTSSQQQNMAAFTALGVEVAITELD
IRMQLPETEALLTQQATDYQSTVQACANTKGCVGITVWDWTDKYSWVP
STFSGYGDACPWDANYQKKPAYEGILTGLGQTVTSTTYIISPTTSVGT
GTTTSSGGSGGTTGVAQHWEQCGGLGWTGPTVCASGYTCTVINEYYSQ
CL
AtuXyn4, Aspergillus tubigensis (SEQ ID NO: 3):
EPIEPRQASVSIDTKFKAHGKKYLGNIGDQYTLTKNSKTPAIIKADFG
ALTPENSMKWDATEPSRGQFSFSGSDYLVNFAQSNNKLIRGHTLVWHS
QLPSWVQSITDKNTLIEVMKNHITTVMQHYKGKIYAWDVVNEIFNEDG
SLRDSVFYKVIGEDYVRIAFETARAADPNAKLYINDYNLDSASYPKLT
GMVSHVKKWIAAGIPIDGIGSQTHLSAGGGAGISGALNALAGAGTKEI
AVTELDIAGASSTDYVEVVEACLNQPKCIGITVWGVADPDSWRSSSTP
LLFDSNYNPKPAYTAIANAL
AacXyn2, Aspergillus aculeatus (SEQ ID NO: 4):
MVGLLSITAALAATVLPNIVSAVGLDQAAVAKGLQYFGTATDNPELTD
IPYVTQLNNTADFGQITPGNSMKWDATEPSQGTFTFTKGDVIADLAEG
NGQYLRCHTLVWYNQLPSWVTSGTWTNATLTAALKNHITNVVSHYKGK
CLHWDVVNEALNDDGTYRTNIFYTTIGEAYIPIAFAAAAAADPDAKLF
YNDYNLEYGGAKAASARAIVQLVKNAGAKIDGVGLQAHFSVGTVPSTS
SLVSVLQSFTALGVEVAYTEADVRILLPTTATTLAQQSSDFQALVQSC
VQTTGCVGFTIWDWTDKYSWVPSTFSGYGAALPWDENLVKKPAYNGLL
AGMGVTVTTTTTTTTATATGKTTTTTTGATSTGTTAAHWGQCGGLNWS
GPTACATGYTCTYVNDYYSQCL
TreXyn3, Trichoderma reesei (SEQ ID NO: 5):
MKANVILCLLAPLVAALPTETIHLDPELAALRANLTERTADLWDRQAS
QSIDQLIKRKGKLYFGTATDRGLLQREKNAAIIQADLGQVTPENSMKW
QSLENNQGQLNWGDADYLVNFAQQNGKSIRGHTLIWHSQLPAWVNNIN
NADTLRQVIRTHVSTVVGRYKGKIRAWDVVNEIFNEDGTLRSSVFSRL
LGEEFVSIAFRAARDADPSARLYINDYNLDRANYGKVNGLKTYVSKWI
SQGVPIDGIGSQSHLSGGGGSGTLGALQQLATVPVTELAITELDIQGA
PTTDYTQVVQACLSVSKCVGITVWGISDKDSWRASTNPLLFDANFNPK
PAYNSIVGILQ
TreXyn5, Trichoderma reesei (SEQ ID NO: 6):
QCIQPGTGYNNGYFYSYWNDGHGGVTYCNGPGGQFSVNWSNSGNFVGG
KGWQPGTKNRVINFSGSYNPNGNSYLSVYGWSRNPLIEYYIVENFGTY
NPSTGATKLGEVTSDGSVYDIYRTQRVNQPSIIGTATFYQYWSVRRNH
RSSGSVNTANHFNAWAQQGLTLGTMDYQIVAVEGYFSSGSASITVSD
BsuGluS, Bacillus subtilis (SEQ ID NO: 7),
214 aa:
QTGGSFFDPFNGYNSGFWQKADGYSNGNMFNCTWRANNVSMTSLGEMR
LALTSPAYNKFDCGENRSVQTYGYGLYEVRMKPAKNTGIVSSFFTYTG
PTDGTPWDEIDIEFLGKDTTKVQFNYYTNGAGNHEKIVDLGFDAANAY
HTYAFDWQPNSIKWYVDGQLKHTATNQIPTTPGKIMMNLWNGTGVDEW
LGSYNGVNPLYAHYDWVRYTKK
TerGlu1, Geosmithia emersonii (Taleromyces
emersonii) (SEQ ID NO: 8):
APVKEKGIKKRASPFQWFGSNESGAEFGNNNIPGVEGTDYTFPNTSAI
QILIDQGMNIFRVPFLMERMVPNQMTGPVDSAYFQGYSQVINYITSHG
ASAVIDPHNFGRYYNNIISSPSDFQTFWHTIASNFADNDNVIFDTNNE
YHDMDESLVVQLNQAAIDGIRAAGATSQYIFVEGNSWTGAWTWTQVND
AMANLTDPQNKIVYEMHQYLDSDGSGTSDQCVNSTIGQDRVESATAWL
KQNGKKAILGEYAGGANSVCETAVTGMLDYLANNTDVWTGAIWWAAGP
WWGDYIFSMEPPSGIAYEQVLPLLQPYL
BsuGlu103FULL, Bacillus subtilis (SEQ ID NO: 9):
DDYSVVEEHGQLSISNGELVNERGEQVQLKGMSSHGLQWYGQFVNYES
MKWLRDDWGITVFRAAMYTSSGGYIDDPSVKEKVKETVEAAIDLGIYV
IIDWHILSDNDPNIYKEEAKDFFDEMSELYGDYPNVIYEIANEPNGSD
VTWDNQIKPYAEEVIPVIRDNDPNNIVIVGTGTWSQDVHHAADNQLAD
PNVMYAFHFYAGTHGQNLRDQVDYALDQGAAIFVSEWGTSAATGDGGV
FLDEAQVWIDFMDERNLSWANWSLTHKDESSAALMPGANPTGGWTEAE
LSPSGTFVREKIRESASIPPSDPTPPSDPGEPDPGEPDPTPPSDPGEY
PAWDSNQIYTNEIVYHNGQLWQAKWWTQNQEPGDPYGPWEPLKSDPDS
GEPDPTPPSDPGEYPAWDSNQIYTNEIVYHNGQLWQAKWWTQNQEPGD
PYGPWEPLN
TreGlu2, Trichoderma reesei (SEQ ID NO: 10):
QQTVWGQCGGIGWSGPTNCAPGSACSTLNPYYAQCIPGATTITTSTRP
PSGPTTTTRATSTSSSTPPTSSGVRFAGVNIAGFDFGCTTDGTCVTSK
VYPPLKNFTGSNNYPDGIGQMQHFVNDDGMTIFRLPVGWQYLVNNNLG
GNLDSTSISKYDQLVQGCLSLGAYCIVDIHNYARWNGGIIGQGGPTNA
QFTSLWSQLASKYASQSRVWFGIMNEPHDVNINTWAATVQEVVTAIRN
AGATSQFISLPGNDWQSAGAFISDGSAAALSQVTNPDGSTTNLIFDVH
KYLDSDNSGTHAECTTNNIDGAFSPLATWLRQNNRQAILTETGGGNVQ
SCIQDMCQQIQYLNQNSDVYLGYVGWGAGSFDSTYVLTETPTGSGNSW
TDTSLVSSCLARK
TreGlu3, Trichoderma reesei (SEQ ID NO: 11):
QTSCDQWATFTGNGYTVSNNLWGASAGSGFGCVTAVSLSGGASWHADW
QWSGGQNNVKSYQNSQIAIPQKRTVNSISSMPTTASWSYSGSNIRANV
AYDLFTAANPNHVTYSGDYELMIWLGKYGDIGPIGSSQGTVNVGGQSW
TLYYGYNGAMQVYSFVAQTNTTNYSGDVKNFFNYLRDNKGYNAAGQYV
LSYQFGTEPFTGSGTLNVASWTASIN
TreGlu4, Trichoderma reesei (SEQ ID NO: 12):
HGHINDIVINGVWYQAYDPTTFPYESNPPIVVGWTAADLDNGFVSPDA
YQNPDIICHKNATNAKGHASVKAGDTILFQWVPVPWPHPGPIVDYLAN
CNGDCETVDKTTLEFFKIDGVGLLSGGDPGTWASDVLISNNNTWVVKI
PDNLAPGNYVLRHEIIALHSAGQANGAQNYPQCFNIAVSGSGSLQPSG
VLGTDLYHATDPGVLINIYTSPLNYIIPGPTVVSGLPTSVAQGSSAAT
ATASATVPGGGSGPTSRTTTTARTTQASSRPSSTPPATTSAPAGGPTQ
TLYGQCGGSGYSGPTRCAPPATCSTNPYYAQCLN
TreGlu6, Trichoderma reesei (SEQ ID NO: 13):
AFSWKNVKLGGGGGFVPGIIFHPKTKGVAYARTDIGGLYRLNADDSWT
AVTDGIADNAGWHNWGIDAVALDPQDDQKVYAAVGMYTNSWDPSNGAI
IRSSDRGATWSFTNLPFKVGGNMPGRGAGERLAVDPANSNIIYFGARS
GNGLWKSTDGGVTFSKVSSFTATGTYIPDPSDSNGYNSDKQGLMWVTF
DSTSSTTGGATSRIFVGTADNITASVYVSTNAGSTWSAVPGQPGKYFP
HKAKLQPAEKALYLTYSWWPDAQLFRSTDSGTTWSPIWAWASYPTETY
YYSISTPKAPWIKNNFIDVTSESPSDGLIKRLGWMIESLEIDPTDSNH
WLYGTGMTIFGGHDLTNWDTRHNVSIQSLADGIEEFSVQDLASAPGGS
ELLAAVGDDNGFTFASRNDLGTSPQTVWATPTWATSTSVDYAGNSVKS
VVRVGNTAGTQQVAISSDGGATWSIDYAADTSMNGGTVAYSADGDTIL
WSTASSGVQRSQFQGSFASVSSLPAGAVIASDKKTNSVFYAGSGSTFY
VSKDTGSSFTRGPKLGSAGTIRDIAAHPTTAGTLYVSTDVGIFRSTDS
GTTFGQVSTALTNTYQIALGVGSGSNWNLYAFGTGPSGARLYASGDSG
ASWTDIQGSQGFGSIDSTKVAGSGSTAGQVYVGTNGRGVFYAQGTVGG
GTGGTSSSTKQSSSSTSSASSSTTLRSSVVSTTRASTVTSSRTSSAAG
PTGSGVAGHYAQCGGIGWTGPTQCVAPYVCQKQNDYYYQCV
TreGlu7, Trichoderma reesei (SEQ ID NO: 14):
HGQVQNFTINGQYNQGFILDYYYQKQNTGHFPNVAGWYAEDLDLGFIS
PDQYTTPDIVCHKNAAPGAISATAAAGSNIVFQWGPGVWPHPYGPIVT
YVVECSGSCTTVNKNNLRWVKIQEAGINYNTQVWAQQDLINQGNKWTV
KIPSSLRPGNYVFRHELLAAHGASSANGMQNYPQCVNIAVTGSGTKAL
PAGTPATQLYKPTDPGILFNPYTTITSYTIPGPALWQG
TreGlu8, Trichoderma reesei (SEQ ID NO: 15):
GKIKYLGVAIPGIDFGCDIDGSCPTDTSSVPLLSYKGGDGAGQMKHFA
EDDGLNVFRISATWQFVLNNTVDGKLDELNWGSYNKVVNACLETGAYC
MIDMHNFARYNGGIIGQGGVSDDIFVDLWVQIAKYYEDNDKIIFGLMN
EPHDLDIEIWAQTCQKVVTAIRKAGATSQMILLPGTNFASVETYVSTG
SAEALGKITNPDGSTDLLYFDVHKYLDINNSGSHAECTTDNVDAFNDF
ADWLRQNKRQAIISETGASMEPSCMTAFCAQNKAISENSDVYIGFVGW
GAGSFDTSYILTLTPLGKPGNYTDNKLMNECILDQFTLDEKYRPTPTS
ISTAAEETATATATSDGDAPSTTKPIFREETASPTPNAVTKPSPDTSD
SSDDDKDSAASMSAQGLTGTVLFTVAALGYMLVAF
BsuGluC CBD, Bacillus subtilis (SEQ ID NO: 16):
MKRSISIFITCLLITLLTMGGMIASPASAAGTKTPVAKNGQLSIKGTQ
LVNRDGKAVQLKGISSHGLQWYGEYVNKDSLKWLRDDWGITVFRAAMY
TADGGYIDNPSVKNKVKEAVEAAKELGIYVIIDWHILNDGNPNQNKEK
AKEFFKEMSSLYGNTPNVIYEIANEPNGDVNWKRDIKPYAEEVISVIR
KNDPDNIIIVGTGTWSQDVNDAADDQLKDANVMYALHFYAGTHGQFLR
DKANYALSKGAPIFVTEWGTSDASGNGGVFLDQSREWLKYLDSKTISW
VNWNLSDKQESSSALKPGASKTGGWRLSDLSASGTFVRENILGTKDST
KDIPETPSKDKPTQENGISVQYRAGDGSMNSNQIRPQLQIKNNGNTTV
DLKDVTARYWYKAKNKGQNFDCDYAQIGCGNVTHKFVTLHKPKQGADT
YLELGFKNGTLAPGASTGNIQLRLHNDDWSNYAQSGDYSFFKSNTFKT
TKKITLYDQGKLIWGTEPN
BsuXyn3, Bacillus subtilis xylanase variant
(SEQ ID NO: 17):
ASTDYWQNWTFGGGIVNAVNGSGGNYSVNWSNTGNFVVGKGWTTGSPF
RTINYNAGVWAPNGNGYLTLYGWTRSPLIEYYVVDSWGTYRPTGTYKG
TVKSDGGTYDIYTTTRYNAPSIDGDDTTFTQYWSVRQSKRPTGSNATI
TFSNHVNAWKSHGMNLGSNWAYQVMATEGYQSSGSSNVTVW
BsuXyn4, Bacillus subtilis xylanase variant
(SEQ ID NO: 18):
ASTDYWQNWTDGYGIVNAVNGSGGNYSVNWSNTGNFVVGKGWTTGSPF
RTINYNAGVWAPNGNGYLTLYGWTRSPLIEYYVVDSWGTYRPTGTYKG
TVYSDGGWYDIYTATRDNAPSIDGDFTTFTQYWSVRQSKRPTGSNATI
TFSNHVNAWRSHGMDLGSNWAYQVMATEGYLSSGSSNVTVW

1. A method of producing malted cereal comprising:
a) providing a cereal grain comprising one or more β-glucans and one or more arabinoxylans,
b) adding to said cereal an effective amount of a β-glucanase and an effective amount of a xylanase during a malting process; and
c) obtaining a malted cereal.

2. The method of any preceding claims, wherein said method comprises a steeping step including one or more wetting stages, where said β-glucanase and/or said xylanase are added one or more times during said one or more of wetting stages.

3. The method of claim 2, wherein:
(i) said β-glucanase and/or said xylanase are added at the beginning of the first wetting of the cereal; or
(ii) said β-glucanase and/or said xylanase are added during the first wetting of the cereal; or
(iii) said β-glucanase and/or said xylanase are added at the beginning of the second wetting of the cereal; or
(iv) said β-glucanase and/or said xylanase are added during the second wetting of the cereal; or
(v) said β-glucanase and/or said xylanase are added at the beginning of the third wetting of the cereal; or
(vi) said β-glucanase and/or said xylanase are added during the third wetting of the cereal; or
(vii) said β-glucanase and/or said xylanase are added at the beginning of all wetting stages of the cereal; or
(viii) said β-glucanase and/or said xylanase are added during all wetting stages of the cereal; or
(ix) said one or more wetting stages comprise spraying the cereal and wherein said 13-glucanase and/or said xylanase are added one or more times, or constantly, during said spraying.

4. The method of any preceding claim, wherein the cereal reaches a moisture content of between about 40 to about 50% W/W, wherein said moisture content is reached in less than 12 hours, and wherein said moisture content is reached with at least 10% less water than the water used for a malted cereal prepared without saidβ-glucanase and/or said xylanase.

5. The method of any preceding claim, wherein:
(i) said cereal has 1-10% W/W of β-glucans; or
(ii) said cereal has 1-10% W/W of arabinoxylans; or
(iii) the amount of high molecular weight β-glucans in the cereal is decreased at least 50%; or
(iv) the amount of high molecular weight β-glucans in the cereal is decreased at least 80%; or
(v) the amount of high molecular weight arabinoxylans in the cereal is decreased at least 50%.; or
(vi) the cereal is selected from the group consisting of barley, wheat, rye, corn, oats, rice, millet, triticale, cassava, sorghum and a combination thereof; or
(vii) said β-glucanase is dosed at 0.01-100 mg enzyme protein per kilogram of cereal; or
(viii) β-glucanase is dosed at 1-15 mg enzyme protein per kilogram of cereal; or
(ix) said xylanase is dosed at 0.01-100 mg enzyme protein per kilogram of cereal; or
(xi) said xylanase is dosed at 1-15 mg enzyme protein per kilogram of cereal.

6. The method of any preceding claim, wherein saidβ-glucanase is an endo-1,3(4)-β-glucanase.

7. The method of claim 6, wherein said endo-1,3(4)-β-glucanase enzyme comprises an amino acid sequence having at least 80% identity with any one selected from SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof

8. The method of claims any preceding claim, wherein said xylanase is an endo-1,4-β-xylanase.

9. The method of claim 8, wherein said endo-1,4-β-xylanase enzyme comprises an amino acid sequence having at least 80% identity with any one selected from SEQ ID NO:1, SEQ ID NO:2, SEQ ID NO:3, SEQ ID NO:4, SEQ ID NO:5, SEQ ID NO:6, SEQ ID NO:17, and SEQ ID NO:18, or any functional fragment thereof

10. The method of any preceding claim, wherein said β-glucanase and said xylanase have an amino acid sequence having at least 80% sequence identity with the respective SEQ ID, or any functional fragment thereof, being selected from the list consisting of SEQ ID NO:1 and SEQ ID NO:7; SEQ ID NO:2 and SEQ ID NO:7; SEQ ID NO:3 and SEQ ID NO:7; SEQ ID NO:4 and SEQ ID NO:7; SEQ ID NO:5 and SEQ ID NO:7; SEQ ID NO:6 and SEQ ID NO:7; SEQ ID NO:17 and SEQ ID NO:7; SEQ ID NO:18 and SEQ ID NO:7; SEQ ID NO:1 and SEQ ID NO:8; SEQ ID NO:2 and SEQ ID NO:8; SEQ ID NO:3 and SEQ ID NO:8; SEQ ID NO:4 and SEQ ID NO:8; SEQ ID NO:5 and SEQ ID NO:8; SEQ ID NO:6 and SEQ ID NO:8; SEQ ID NO:17 and SEQ ID NO:8; SEQ ID NO:18 and SEQ ID NO:8; SEQ ID NO:1 and SEQ ID NO:9; SEQ ID NO:2 and SEQ ID NO:9; SEQ ID NO:3 and SEQ ID NO:9; SEQ ID NO:4 and SEQ ID NO:9; SEQ ID NO:5 and SEQ ID NO:9; SEQ ID NO:6 and SEQ ID NO:9; SEQ ID NO:17 and SEQ ID NO:9; SEQ ID NO:18 and SEQ ID NO:9; SEQ ID NO:1 and SEQ ID NO:10; SEQ ID NO:2 and SEQ ID NO:10; SEQ ID NO:3 and SEQ ID NO:10; SEQ ID NO:4 and SEQ ID NO:10; SEQ ID NO:5 and SEQ ID NO:10; SEQ ID NO:6 and SEQ ID NO:10; SEQ ID NO:17 and SEQ ID NO:10; SEQ ID NO:18 and SEQ ID NO:10; SEQ ID NO:1 and SEQ ID NO:11; SEQ ID NO:2 and SEQ ID NO:11; SEQ ID NO:3 and SEQ ID NO:11; SEQ ID NO:4 and SEQ ID NO:11; SEQ ID NO:5 and SEQ ID NO:11; SEQ ID NO:6 and SEQ ID NO:11; SEQ ID NO:17 and SEQ ID NO:11; SEQ ID NO:18 and SEQ ID NO:11; SEQ ID NO:1 and SEQ ID NO:12; SEQ ID NO:2 and SEQ ID NO:12; SEQ ID NO:3 and SEQ ID NO:12; SEQ ID NO:4 and SEQ ID NO:12; SEQ ID NO:5 and SEQ ID NO:12; SEQ ID NO:6 and SEQ ID NO:12; SEQ ID NO:17 and SEQ ID NO:12; SEQ ID NO:18 and SEQ ID NO:12; SEQ ID NO:1 and SEQ ID NO:13; SEQ ID NO:2 and SEQ ID NO:13; SEQ ID NO:3 and SEQ ID NO:13; SEQ ID NO:4 and SEQ ID NO:13; SEQ ID NO:5 and SEQ ID NO:13; SEQ ID NO:6 and SEQ ID NO:13; SEQ ID NO:17 and SEQ ID NO:13; SEQ ID NO:18 and SEQ ID NO:13; SEQ ID NO:1 and SEQ ID NO:14; SEQ ID NO:2 and SEQ ID NO:14; SEQ ID NO:3 and SEQ ID NO:14; SEQ ID NO:4 and SEQ ID NO:14; SEQ ID NO:5 and SEQ ID NO:14; SEQ ID NO:6 and SEQ ID NO:14; SEQ ID NO:17 and SEQ ID NO:14; SEQ ID NO:18 and SEQ ID NO:14; SEQ ID NO:1 and SEQ ID NO:15; SEQ ID NO:2 and SEQ ID NO:15; SEQ ID NO:3 and SEQ ID NO:15; SEQ ID NO:4 and SEQ ID NO:15; SEQ ID NO:5 and SEQ ID NO:15; SEQ ID NO:6 and SEQ ID NO:15; SEQ ID NO:17 and SEQ ID NO:15; SEQ ID NO:18 and SEQ ID NO:15; SEQ ID NO:1 and SEQ ID NO:16; SEQ ID NO:2 and SEQ ID NO:16; SEQ ID NO:3 and SEQ ID NO:16; SEQ ID NO:4 and SEQ ID NO:16; SEQ ID NO:5 and SEQ ID NO:16; SEQ ID NO:6 and SEQ ID NO:16; SEQ ID NO:17 and SEQ ID NO:16; and SEQ ID NO:18 and SEQ ID NO:16.

11. The method of any preceding claim, wherein said β-glucanase and said xylanase have an amino acid sequence having at least 80% sequence identity with the respective SEQ ID, or any functional fragment thereof, being selected from the list consisting of SEQ ID NO:1 and SEQ ID NO:7; SEQ ID NO:2 and SEQ ID NO:8; and SEQ ID NO:2 and SEQ ID NO:8.

12. The method of any preceding claim, wherein:
(i) said xylanase and said beta-glucanase are combined at a ratio of 1:1 per weight; or
(ii) said beta-glucanase has a beta-glucanase activity of at least 9000 U/g, and said xylanase has a xylanase activity of at least 13000U/g; or
(iii) said β-glucanase and said xylanase comprises commercially available enzymes having xylanase activity and/or beta-glucanase activity selected from the group consisting of UltraFlo L (available from Novozymes-beta glucanase with cellulase, xylanase side activities), UltraFlo XL (available from Novozymes-beta glucanase with xylanase and alpha amylase side activities), UltraFlo Max (available from Novozymes-beta glucanase and xylanase), Finizyme 250 L(available from Novozymes-beta glucanase with cellulase, xylanase side activities) and Filtrase series(available from DSM-betaglucanase and xylanases); or
(iv) a total steeping time is at least 10% less than the time of a malted cereal prepared without said β-glucanase and/or said xylanase; or
(v) the total steeping time does not exceed 25 hours; or
(vi) the total steeping time does not exceed 12 hours; or
(vii) the total amount of water used during the steeping process is at least 10% less than the amount of water used for a malted cereal prepared without said β-glucanase and/or said xylanase; or
(viii) the amount of high molecular weight β-glucans in the malted cereal is at least 10% less than the amount of high molecular weight β-glucans in a malted cereal prepared without saidβ-glucanase; or
(ix) the amount of high molecular weight arabinoxylans is at least 10% less than the amount of arabinoxylans in a malted cereal prepared without said xylanase; or
(x) the amount of high molecular weight β-glucans and/or arabinoxylans is at least 50% less than the amount of β-glucans and/or arabinoxylans in a malted cereal prepared without said β-glucanase and/or said xylanase; or
(xi) the amount of high molecular weight β-glucans in said malted cereal is 50 mg/l or less, and the amount of arabinoxylans in said malted cereal is at 2000 mg/l or less; or
(xii) an extract prepared from said malted cereal has lower viscosity that an extract prepared with a malted cereal prepared without saidβ-glucanase and/or said xylanase; or
(xiii) the malted cereal has increased enzymatic activity.

13. The method of claim 12 (xiii), wherein:
(a) said enzymatic activity is increased by a factor of at least about 2 as compared to a malted cereal prepared without said β-glucanase and/or said xylanase; or
(b) said enzymatic activity is selected from the group consisting ofβ-glucanase activity, xylanase activity, amylase activity, protease activity, naturally occurring enzyme activity in the cereal and combinations thereof; or
(c) said enzymatic activity is β-glucanase and/or xylanase activity.

14. The method according to any preceding claim, further comprising adding one or more enzymes selected from the group of amylase, a protease, naturally occurring enzymes in the cereal and combinations thereof.

15. The method according to any preceding claim, said method comprises a steeping step including one or more wetting stages, where said β-glucanase and/or said xylanase are added one or more times during said one or more of wetting stages, and wherein the amount of high molecular weight β-glucans in said malted cereal is 50 mg/l or less, and the amount of arabinoxylans in said malted cereal is at 2000 mg/l or less.


SEQUENCE LISTING (18 sequences)

SEQ ID NO: 1 - protein, 302 aa, Aspergillus tubigensis
QASVSIDTKFKAHGKKYLGNIGDQYTLTKNSKTPAIIKADFGALTPENSMKWDATEPSRGQFSFSGSDYLVNFAQSNNKLIRGHTLVWHSQLPSWVQAITDKNTLIEVMKNHITTVMQHYKGKIYAWDVVNEIFNEDGSLRDSVFYQVIGEDYVRIAFETARAADPNAKLYINDYNLDSASYPKLTGMVSHVKKWIEAGIPIDGIGSQTHLSAGGGAGISGALNALAGAGTKEIAVTELDIAGASSTDYVEVVEACLDQPKCIGITVWGVADPDSWRSSSTPLLFDSNYNPKPAYTAIANAL

SEQ ID NO: 2 - protein, 386 aa, Geosmithia emersonii
AGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 3 - protein, 308 aa, Aspergillus tubigensis
EPIEPRQASVSIDTKFKAHGKKYLGNIGDQYTLTKNSKTPAIIKADFGALTPENSMKWDATEPSRGQFSFSGSDYLVNFAQSNNKLIRGHTLVWHSQLPSWVQSITDKNTLIEVMKNHITTVMQHYKGKIYAWDVVNEIFNEDGSLRDSVFYKVIGEDYVRIAFETARAADPNAKLYINDYNLDSASYPKLTGMVSHVKKWIAAGIPIDGIGSQTHLSAGGGAGISGALNALAGAGTKEIAVTELDIAGASSTDYVEVVEACLNQPKCIGITVWGVADPDSWRSSSTPLLFDSNYNPKPAYTAIANAL

SEQ ID NO: 4 - protein, 406 aa, Aspergillus aculeatus
MVGLLSITAALAATVLPNIVSAVGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 5 - protein, 347 aa, Trichoderma reesei
MKANVILCLLAPLVAALPTETIHLDPELAALRANLTERTADLWDRQASQSIDQLIKRKGKLYFGTATDRGLLQREKNAAIIQADLGQVTPENSMKWQSLENNQGQLNWGDADYLVNFAQQNGKSIRGHTLIWHSQLPAWVNNINNADTLRQVIRTHVSTVVGRYKGKIRAWDVVNEIFNEDGTLRSSVFSRLLGEEFVSIAFRAARDADPSARLYINDYNLDRANYGKVNGLKTYVSKWISQGVPIDGIGSQSHLSGGGGSGTLGALQQLATVPVTELAITELDIQGAPTTDYTQVVQACLSVSKCVGITVWGISDKDSWRASTNPLLFDANFNPKPAYNSIVGILQ

SEQ ID NO: 6 - protein, 191 aa, Trichoderma reesei
QCIQPGTGYNNGYFYSYWNDGHGGVTYCNGPGGQFSVNWSNSGNFVGGKGWQPGTKNRVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVSD

SEQ ID NO: 7 - protein, 214 aa, Bacillus subtilis
QTGGSFFDPFNGYNSGFWQKADGYSNGNMFNCTWRANNVSMTSLGEMRLALTSPAYNKFDCGENRSVQTYGYGLYEVRMKPAKNTGIVSSFFTYTGPTDGTPWDEIDIEFLGKDTTKVQFNYYTNGAGNHEKIVDLGFDAANAYHTYAFDWQPNSIKWYVDGQLKHTATNQIPTTPGKIMMNLWNGTGVDEWLGSYNGVNPLYAHYDWVRYTKK

SEQ ID NO: 8 - protein, 316 aa, Geosmithia emersonii
APVKEKGIKKRASPFQWFGSNESGAEFGNNNIPGVEGTDYTFPNTSAIQILIDQGMNIFRVPFLMERMVPNQMTGPVDSAYFQGYSQVINYITSHGASAVIDPHNFGRYYNNIISSPSDFQTFWHTIASNFADNDNVIFDTNNEYHDMDESLVVQLNQAAIDGIRAAGATSQYIFVEGNSWTGAWTWTQVNDAMANLTDPQNKIVYEMHQYLDSDGSGTSDQCVNSTIGQDRVESATAWLKQNGKKAILGEYAGGANSVCETAVTGMLDYLANNTDVWTGAIWWAAGPWWGDYIFSMEPPSGIAYEQVLPLLQPYL

SEQ ID NO: 9 - protein, 441 aa, Bacillus subtilis
DDYSVVEEHGQLSISNGELVNERGEQVQLKGMSSHGLQWYGQFVNYESMKWLRDDWGITVFRAAMYTSSGGYIDDPSVKEKVKETVEAAIDLGIYVIIDWHILSDNDPNIYKEEAKDFFDEMSELYGDYPNVIYEIANEPNGSDVTWDNQIKPYAEEVIPVIRDNDPNNIVIVGTGTWSQDVHHAADNQLADPNVMYAFHFYAGTHGQNLRDQVDYALDQGAAIFVSEWGTSAATGDGGVFLDEAQVWIDFMDERNLSWANWSLTHKDESSAALMPGANPTGGWTEAELSPSGTFVREKIRESASIPPSDPTPPSDPGEPDPGEPDPTPPSDPGEYPAWDSNQIYTNEIVYHNGQLWQAKWWTQNQEPGDPYGPWEPLKSDPDSGEPDPTPPSDPGEYPAWDSNQIYTNEIVYHNGQLWQAKWWTQNQEPGDPYGPWEPLN

SEQ ID NO: 10 - protein, 397 aa, Trichoderma reesei
QQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 11 - protein, 218 aa, Trichoderma reesei
QTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 12 - protein, 322 aa, Trichoderma reesei
HGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTNPYYAQCLN

SEQ ID NO: 13 - protein, 761 aa, Trichoderma reesei
AFSWKNVKLGGGGGFVPGIIFHPKTKGVAYARTDIGGLYRLNADDSWTAVTDGIADNAGWHNWGIDAVALDPQDDQKVYAAVGMYTNSWDPSNGAIIRSSDRGATWSFTNLPFKVGGNMPGRGAGERLAVDPANSNIIYFGARSGNGLWKSTDGGVTFSKVSSFTATGTYIPDPSDSNGYNSDKQGLMWVTFDSTSSTTGGATSRIFVGTADNITASVYVSTNAGSTWSAVPGQPGKYFPHKAKLQPAEKALYLTYSWWPDAQLFRSTDSGTTWSPIWAWASYPTETYYYSISTPKAPWIKNNFIDVTSESPSDGLIKRLGWMIESLEIDPTDSNHWLYGTGMTIFGGHDLTNWDTRHNVSIQSLADGIEEFSVQDLASAPGGSELLAAVGDDNGFTFASRNDLGTSPQTVWATPTWATSTSVDYAGNSVKSVVRVGNTAGTQQVAISSDGGATWSIDYAADTSMNGGTVAYSADGDTILWSTASSGVQRSQFQGSFASVSSLPAGAVIASDKKTNSVFYAGSGSTFYVSKDTGSSFTRGPKLGSAGTIRDIAAHPTTAGTLYVSTDVGIFRSTDSGTTFGQVSTALTNTYQIALGVGSGSNWNLYAFGTGPSGARLYASGDSGASWTDIQGSQGFGSIDSTKVAGSGSTAGQVYVGTNGRGVFYAQGTVGGGTGGTSSSTKQSSSSTSSASSSTTLRSSVVSTTRASTVTSSRTSSAAGPTGSGVAGHYAQCGGIGWTGPTQCVAPYVCQKQNDYYYQCV

SEQ ID NO: 14 - protein, 230 aa, Trichoderma reesei
HGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 15 - protein, 419 aa, Trichoderma reesei
GKIKYLGVAIPGIDFGCDIDGSCPTDTSSVPLLSYKGGDGAGQMKHFAEDDGLNVFRISATWQFVLNNTVDGKLDELNWGSYNKVVNACLETGAYCMIDMHNFARYNGGIIGQGGVSDDIFVDLWVQIAKYYEDNDKIIFGLMNEPHDLDIEIWAQTCQKVVTAIRKAGATSQMILLPGTNFASVETYVSTGSAEALGKITNPDGSTDLLYFDVHKYLDINNSGSHAECTTDNVDAFNDFADWLRQNKRQAIISETGASMEPSCMTAFCAQNKAISENSDVYIGFVGWGAGSFDTSYILTLTPLGKPGNYTDNKLMNECILDQFTLDEKYRPTPTSISTAAEETATATATSDGDAPSTTKPIFREETASPTPNAVTKPSPDTSDSSDDDKDSAASMSAQGLTGTVLFTVAALGYMLVAF

SEQ ID NO: 16 - protein, 499 aa, Bacillus subtilis
MKRSISIFITCLLITLLTMGGMIASPASAAGTKTPVAKNGQLSIKGTQLVNRDGKAVQLKGISSHGLQWYGEYVNKDSLKWLRDDWGITVFRAAMYTADGGYIDNPSVKNKVKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMYALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPSKDKPTQENGISVQYRAGDGSMNSNQIRPQLQIKNNGNTTVDLKDVTARYWYKAKNKGQNFDCDYAQIGCGNVTHKFVTLHKPKQGADTYLELGFKNGTLAPGASTGNIQLRLHNDDWSNYAQSGDYSFFKSNTFKTTKKITLYDQGKLIWGTEPN

SEQ ID NO: 17 - protein, 185 aa, Artificial sequence
  Bacillus subtilis xylanase variant
ASTDYWQNWTFGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDDTTFTQYWSVRQSKRPTGSNATITFSNHVNAWKSHGMNLGSNWAYQVMATEGYQSSGSSNVTVW

SEQ ID NO: 18 - protein, 185 aa, Artificial sequence
  Bacillus subtilis xylanase variant
ASTDYWQNWTDGYGIVNAVNGSGGNYSVNWSNTGNFVVGKGWTTGSPFRTINYNAGVWAPNGNGYLTLYGWTRSPLIEYYVVDSWGTYRPTGTYKGTVYSDGGWYDIYTATRDNAPSIDGDFTTFTQYWSVRQSKRPTGSNATITFSNHVNAWRSHGMDLGSNWAYQVMATEGYLSSGSSNVTVW